(19) United States
(12) Patent Application Publication
(10) Pub. No.: US 2013/0053347 A1
(43) Pub. Date: Feb. 28, 2013

(54) PRO-DRUGS OF AMARYLLIDACEAE ISOCARBOSTYRIL PRODUCTS AND THEIR USE AGAINST BRAIN TUMORS

(71) Applicants: Veronique Mathieu, Brussels (BE); Gwendoline Van Goietsenoven, Fosses-la-Ville (BE); Jacques Dubois, Villers-la-Ville (BE); Florence Lefranc-Kiss, Sint-Pieters-Leeuw (BE); Laurent Ingrassia, Braine I'Alleud (BE); Robert Kiss, Sint-Pieters-Leeuw (BE)

(72) Inventors: Veronique Mathieu, Brussels (BE); Gwendoline Van Goietsenoven, Fosses-la-Ville (BE); Jacques Dubois, Villers-la-Ville (BE); Florence Lefranc-Kiss, Sint-Pieters-Leeuw (BE); Laurent Ingrassia, Braine I'Alleud (BE); Robert Kiss, Sint-Pieters-Leeuw (BE)

(73) Assignee: Universite Libre de Bruxelles, Bruxelles (BE)

(21) Appl. No.: 13/695,609

(22) PCT Filed: May 3, 2010
(86) PCT No.: PCT/EP10/55972

§ 371 Date: Oct. 31, 2012

(51) Int. Cl. C07F 9/6574; G01N 33/566; A61P 35/00; A61K 31/675

(52) U.S. Cl. 514/81; 546/23; 544/8; 540/487; 544/214; 435/7.23; 436/501

(57) ABSTRACT

The present invention relates to the biology and mechanism of action of the naturally occurring compound Narciclasine, especially as an anti-cancer agent for brain tumors. The invention provides new insights on the target molecule of Amaryllidaceae isocarbostyril derivatives as for example Narciclasine and provides new prodrugs of these Amaryllidceae isocabostyril constituents for treating cancer, specifically cancers or tumors located in the brain.

### FIELD OF THE INVENTION

The present invention is situated in the field of anti-cancer drug development and anti-cancer treatment, more specifically towards tumors and cancers located in the brain.

### BACKGROUND OF THE INVENTION

For over two thousand years plants belonging to the Amaryllidaceae family have been used in traditional medicine throughout the world for various anticancer applications. Over 100 alkaloids and isocarbostyril constituents, exhibiting diverse biological activities, have been recently isolated from various Amaryllidaceae species (Kornienko A et al., Chem Rev 2008; 108:1982-2014). Lycorine was the first alkaloid isolated from these plants (Gheorghiu A et al., Ann Pharm Fr 1962; 20:531-8) and found to possess anti-cancer activities both in vitro and in vivo (Liu J et al., Biomed Pharmacother 2007; 61:229-34; Liu X S et al., Cancer Lett 2009; 274:16-24; Lamoral-Theys D et al., J Med Chem 2009; 52: 6244-6256). More recently several isocarbostyril constituents of the Amaryllidaceae (further denominated Al), such as narciclasine, pancratistatin, trans-dihydronarciclasine and their congeners (for structure examples, see below), have been isolated or hemi-synthesized (Pettit G R et al., J Nat Prod 1986; 49:995-1002; Pettit G R et al., J Nat Prod 2009; 72:1279-82). Examples of Amaryllidaceae Isocarbostyril constituents extracted from Amaryllidaceae plants are as follows:

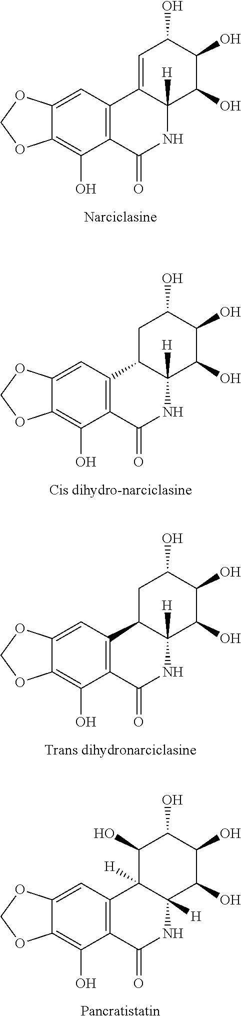

Due to their nanomolar antiproliferative potencies against cancer cells, Al compounds are widely believed to be the most important metabolites responsible for the therapeutic benefits of Amaryllidaceae plants in the folk medical treatment of cancer (Kornienko A et al., Chem Rev 2008; 108:1982-2014; Pettit G R et al., J Nat Prod 1986; 49:995-1002; Pettit G R et al., J Nat Prod 2009; 72:1279-82). Narciclasine, known for more than 40 years and isolated from narcissus bulbs, was first known for its anti-mitotic effects (Ceriotti G. Nature 1967; 213:595-6). Pancratistatin, whose chemical structure is similar to that of narciclasine, induces rapid apoptosis in neuroblastoma cells, but not in normal cells, via mitochondrial targeting (McLachlan A et al., Apoptosis 2005; 10:619-30) that could further synergize with other anti-cancer agents such as tamoxifen (Siedlakowski P et al., Cancer Biol Ther 2008; 7:376-84). Comparable apoptosis induction has been also described for narciclasine in carcinoma cancer cells (Dumont P et al., Neoplasia 2007; 9:766-76).

Narciclasine and the other natural Al compounds thus were initially considered to be of particular interest in anti-cancer treatment. However, the maximal dose of Narciclasine that can be administered to mice without being toxic is around 1 mg/kg. Since a dose of 1 mg/kg has no anti-tumor effect, and the administration of higher doses is toxic, most studies for using Narciclasine as an anti-tumor agent and improving its bioavailability characteristics (e.g. rendering it more soluble, using a slow release formulation etc.) were terminated without much success (see the National Cancer Institute (NCl, USA) database.

In addition, the exact targeting mechanism of Narciclasine to tumor cells has not yet been revealed. Although it has been shown that Al inhibit the peptide bond formation step and thus protein synthesis (Carrasco L et al., FEBS Lett 1975; 52:236-9; Jimenez A et al., Biochim Biophys Acta 1976; 425:342-8), recent data obtained in glioblastoma and prostate cancer cells indicate that Narciclasine could possibly target the actin cytoskeleton and related proteins (Lefranc F et al., Mol Cancer Ther 2009; 8:1739-50; Ingrassia L et al., J Med Chem 2009; 52:1100-14).

The present invention provides new insights in the biology of Amaryllidaceae isocarbostyril constituents and tumor targeting and their potential uses as anti-tumor agents especially for targeting all types of tumors located in the brain.

### SUMMARY OF THE INVENTION

The present invention unravels the mechanism of action of the natural compound Narciclasine, by first identifying its target in tumor cells, namely the eukaryotic elongation factor 1 alpha (eEF1A), whose isoforms are differentially expressed in brain tumors as compared to normal brain tissues. The identification of the target of Narciclasine opens interesting routes for identifying new anti-tumor agents and has importance in cancer diagnosis.

In this respect, the invention provides for a method of screening or identifying new anti-cancer agents comprising the steps of:

a) contacting a candidate anti-cancer agent with the eEF1A receptor,
b) evaluating whether or not said candidate anti-cancer agent binds the eEF1A receptor, and
c) optionally analyzing the effect of the candidate agent on the activity of said eEF1A receptor.

In a preferred embodiment, the activity of the receptor is evaluated by analyzing the growth or progression of said tumor cell.

In addition, the inventors unexpectedly found that Narciclasine had an increased anti-tumor effect on tumors located inside the brain, compared to tumors located outside the brain.

Together with the identification of the receptor of Narciclasine, whose isoforms are differentially expressed in brain tumors as compared to normal brain tissues, the inventors embarked on a new strategy of producing Narciclasine derivatives and others Amaryllidaceae isocarbostyril constituents with improved anti-cancer effect on tumors located in the brain. To this end, two aspects of the Narciclasine compound and others Amaryllidaceae isocarbostyril constituents were taken into consideration:

1) the ability of crossing the blood-brain-barrier (BBB) and
2) the ability to accumulate in the brain, in order to achieve a local (i.e. in the CNS [Central Nervous System] or brain) increase in the dose of Narciclasine or others Amaryllidaceae isocarbostyril constituents in the brain, needed for achieving effective anti-tumor effects in the brain.

The inventors found that synthesising new Narciclasine pro-drugs or other prodrugs of Amaryllidaceae isocarbostyril derivatives that were more lipophillic than the already known natural products and prodrugs, resulted in an increased retention of the active compound into the brain, while retaining an acceptable crossing of the BBB. This is in high contrast to other groups that tried to make these natural products more soluble (hydrophilic) (cf. Pettit and Melody, J. Nat. Prod. 2005, 68:207-211 and WO2004/052298).

The invention thus provides prodrugs of Amaryllidaceae isocarbostyril that are lipophillic and their use in the preparation of a medicament or pharmaceutical composition for treating cancer and tumors located in the CNS.

In a preferred embodiment, the pro-drugs of Amaryllidaceae isocarbostyril derivatives of the invention have a log P value≧1.5 and <3, preferably of about 2.

The invention especially provides pro-drugs of Amaryllidaceae isocarbostyril derivatives of the following general formula (I):

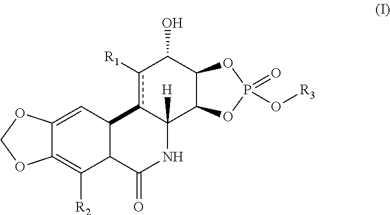

wherein the dotted line can be a single or double bond as exemplified by subgroups of general formulas (Ia) and (Ib):

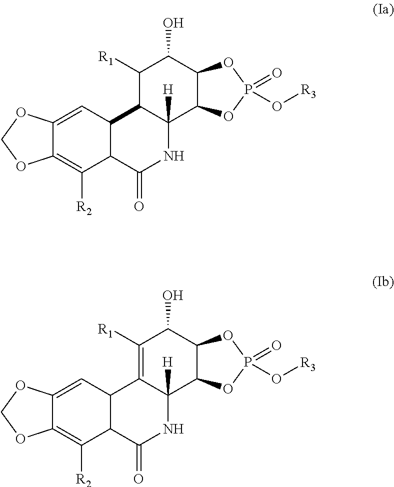

wherein in any one of the formulas I, Ia or Ib:
R₁ is selected from the group comprising: H, OH, OR₄, NR₅R₆, alkyl, aryl, cycloC₁-C₆alkyl, cyclohexyl;
R₂ is selected from the group comprising: H, or OR₇;
R₃ is selected from the group comprising: a substituted or non-substituted straight or branched chain C₁-C₂₄alkyl group, a substituted or non-substituted aryl group, preferably substituted or non-substituted C₆-C₁₈aryl group, a substituted or non-substituted heteroaryl group, a substituted or non-substituted arylalkyl group, preferably a C₆-C₁₈arylC₁-C₆alkyl group, a cycloC₁-C₆alkyl group, a polyoxyalkylene chain of the formula (CxHyO)p, where x is 3 or more carbon atoms, p is 2 or more, and y is from 2x−2 to 2x, a heterocyclyl-C₁-C₂₄alkyl group or a heteroaryl-C₁-C₂₄alkyl, a —C₃-C₂₄alkynyl group, or a —C₃-C₂₄alkenyl group;
R₄, R₅, R₆ and R₇ are each independently selected from the group comprising: H, C₁-C₆alkyl, C₆-C₁₈aryl, carbonyl-C₁-C₆alkyl; carbonyl-C₆-C₁₀aryl;
each group optionally being substituted by a C₁-C₆-alkyl, OXO or ═S.

In a further embodiment, the invention provides pro-drugs of Amaryllidaceae isocarbostyril derivatives of the following general formula (Ic) or (Id):

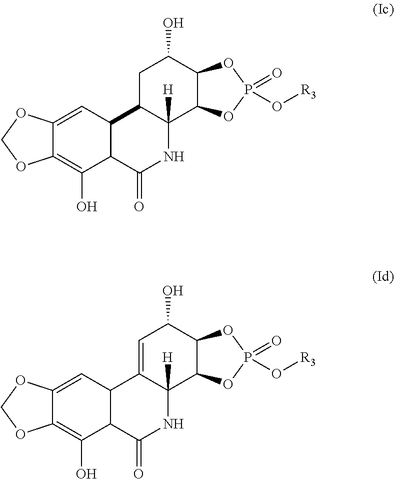

wherein
R₃ is selected from the group comprising: a substituted or non-substituted straight or branched chain C₁-C₂₄alkyl group, a substituted or non-substituted aryl group, preferably substituted or non-substituted C₆-C₁₈aryl group, a substituted or non-substituted heteroaryl group, a substituted or non-substituted arylalkyl group, preferably a C₆-C₁₈arylC₁-C₆alkyl group, a cycloC₁-C₆alkyl group, a polyoxyalkylene chain of the formula (CxHyO)p, where x is 3 or more carbon atoms, p is 2 or more, and y is from 2x−2 to 2x, a heterocyclyl-C₁-C₂₄alkyl group or a heteroaryl-C₁-C₂₄alkyl, a —C₃-C₂₄alkynyl group, or a —C₃-C₂₄alkenyl; each group optionally being substituted by a C₁-C₆-alkyl, *=O (OXO-group) or *=S (thione group). The asterisk (*) is used herein to indicate the point at which a mono- or bivalent radical depicted is connected to the structure to which it relates and of which the radical forms part.

In a preferred embodiment of the pro-drugs of Amaryllidaceae isocarbostyril derivatives of the invention, R₃=a linear C₁-C₂₄-alkyl, such as n-Methyl, n-Ethyl, n-Propyl, n-Butyl, n-Pentyl, n-Hexyl, n-Heptyl, n-Octyl, n-Nonyl, n-Decyl, n-Undecyl, n-Dodecyl, etc.

More preferably, R₃ is C₇H₁₅ as in Formula (Ie) and (If):

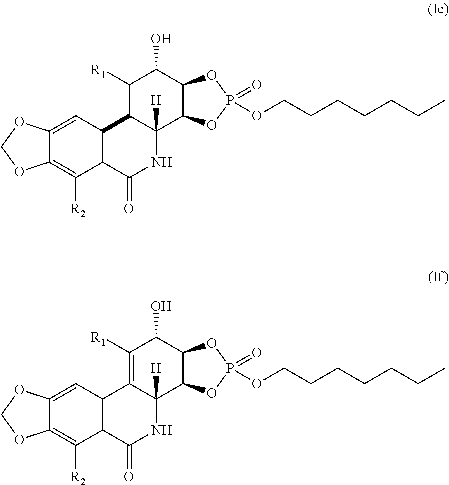

wherein
R₁ and R₂ are each independently selected from the group comprising: H, OH, O—C₁-C₆alkyl.

In a preferred embodiment of the compounds according to formulas Ie and If, R₁ is H and R₂ is OH, leading to compounds N1 (for formula Ie) and N2 (for formula If) as depicted in Table 1.

In a further preferred embodiment of the compounds according to formulas Ia and Ib, R₁ is H and R₂ is OH, and R₃ is phenyl, leading to compounds N3 (for formula Ia) and N4 (for formula Ib) as depicted in Table 1.

In a further preferred embodiment of the compounds according to formulas Ia and Ib, R₁ is H and R₂ is OH, and R₃ is trimethylphenyl, leading to compounds N29 (for formula Ia) and N30 (for formula Ib) as depicted in Table 1.

In a further embodiment, the invention provides pro-drugs of Amaryllidaceae isocarbostyril derivatives of the following general formula (Ig) or (Ih):

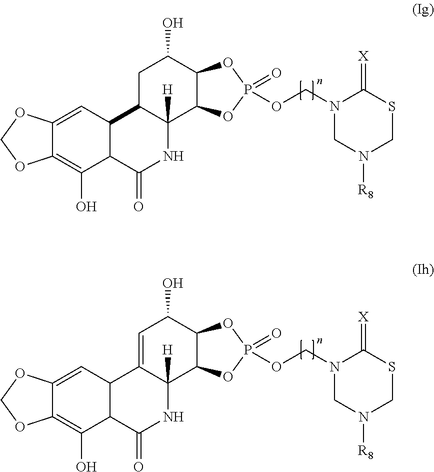

wherein
X is selected from O or S;
n is an integer selected from: 1, 2, 3, 4, 5, 6, 7, 8, 9, 10, 11, 12, 13, 14, 15, 16, 17, 18, 19, 20, 21, 22, 23, or 24.
R₈ is selected from the group comprising: H, C₁-C₆alkyl.

In a more preferred embodiment of formulas Ig and Ih, R₈═CH₃, and X═S. In an even more preferred embodiment of formulas Ig and Ih, R₈═CH₃, X═S, and n=1, leading respectfully to compounds N5 (for formula Ig) and N6 (for formula Ih) in Table 1.

In a further preferred embodiment of the pro-drugs of Amaryllidaceae isocarbostyril derivatives of the invention is as in Formula (II) or (Iⱼ):

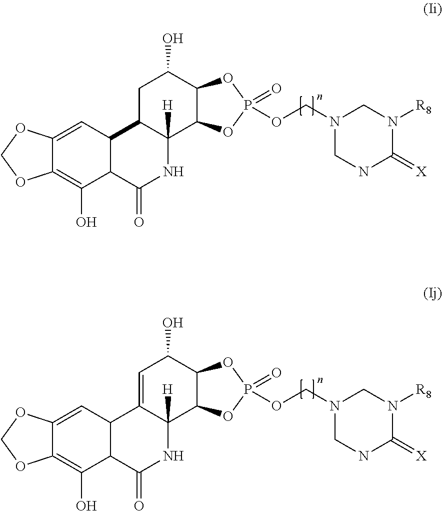

wherein
X is selected from O or S;
n is an integer selected from: 1, 2, 3, 4, 5, 6, 7, 8, 9, 10, 11, 12, 13, 14, 15, 16, 17, 18, 19, 20, 21, 22, 23, or 24.
R₈ is selected from the group comprising: H, C₁-C₆alkyl.

In a preferred embodiment of formulas Ii and Ij, X is S and R₈ is CH₃.

In an even more preferred embodiment of formulas Ii and lj, X═S, R₈═CH₃, and n=1, leading respectively to compounds N13 (for formula Ii) and N14 (for formula Ij) depicted in Table 1.

In a further preferred embodiment of the pro-drugs of Amaryllidaceae isocarbostyril derivatives of the invention is as in Formula (Ik) or (Il):

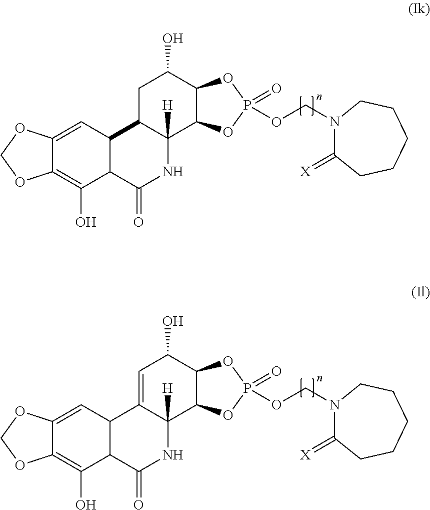

wherein
X is selected from O or S;
n is an integer selected from: 1, 2, 3, 4, 5, 6, 7, 8, 9, 10, 11, 12, 13, 14, 15, 16, 17, 18, 19, 20, 21, 22, 23, or 24.

In a preferred embodiment of formulas Ik and Il, X=O.

In an even more preferred embodiment of formulas Ik and Il, X=O and n=1, leading respectively to compounds N21 (for formula Ik) and N22 (for formula Il).

The invention especially also provides pro-drugs of Amaryllidaceae isocarbostyril derivatives of the following general formula (II):

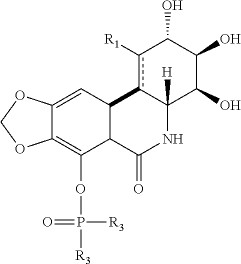

wherein the dotted line can be a single or double bond as exemplified by subgroups of general formulas (IIa) and (IIb):

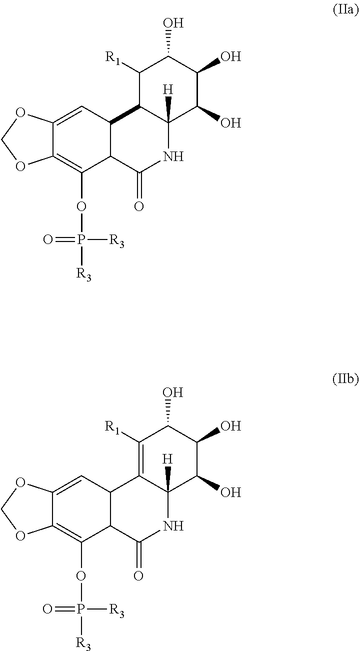

wherein in any one of the formulas II, IIa or IIb:
R₁ is selected from the group comprising: H, OH, OR₄, NR₅R₆, alkyl, aryl, cycloC₁-C₆alkyl, cyclohexyl;

R₃ is independently selected from the group comprising: H, OH, a substituted or non-substituted straight or branched chain O—C₁-C₂₄alkyl, a substituted or non-substituted O-aryl group, preferably substituted or non-substituted O—C₆-C₁₈aryl, a substituted or non-substituted O-heteroaryl group, a substituted or non-substituted O-aryl-alkyl group, preferably a O—C₆-C₁₈arylC₁-C₆alkyl group, a oxy-cycloC₁-C₆alkyl group, a O-polyoxyalkylene chain of the formula O—(CxHyO)p, where x is 3 or more carbon atoms, p is 2 or more, and y is from 2x−2 to 2x, a O-heterocyclyl-C₁-C₂₄alkyl group, a O-heteroaryl-C₁-C₂₄alkyl group, a O—C₃-C₂₄alkynyl group, or a O—C₃-C₂₄alkenyl group;

R₄, R₅, R₆ and R₇ are each independently selected from the group comprising: H, C₁-C₆alkyl, C₆-C₁₈aryl, carbonyl-C₁-C₆alkyl; carbonyl-C₆-C₁₀aryl;
each group optionally being substituted by a C₁-C₆-alkyl, OXO or ═S.

In a further embodiment, the invention provides pro-drugs of Amaryllidaceae isocarbostyril derivatives of the following general formula (IIc) or (IId):

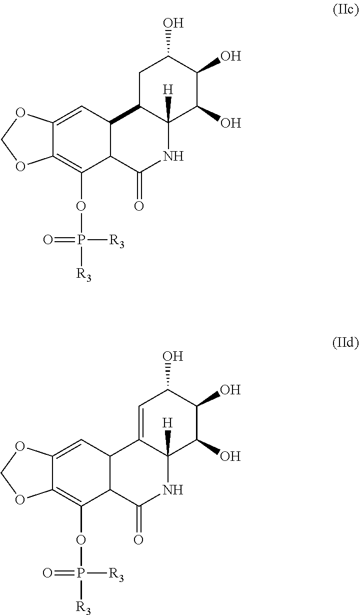

wherein
R₃ is independently selected from the group comprising: H, OH, a substituted or non-substituted straight or branched chain O—C₁-C₂₄alkyl, a substituted or non-substituted O-aryl group, preferably substituted or non-substituted O—C₆-C₁₈aryl, a substituted or non-substituted O-heteroaryl group, a substituted or non-substituted O-aryl-alkyl group, preferably a O—C₆-C₁₈arylC₁-C₆alkyl group, a oxy-cycloC₁-C₆alkyl group, a O-polyoxyalkylene chain of the formula O—(CxHyO)p, where x is 3 or more carbon atoms, p is 2 or more, and y is from 2x−2 to 2x, a O-heterocyclyl-C₁-C₂₄alkyl group, a O-heteroaryl-C₁-C₂₄alkyl group, a O—C₃-C₂₄alkynyl group, or a O—C₃-C₂₄alkenyl group;
each group optionally being substituted by a C₁-C₆-alkyl, *=O (OXO-group) or *=S (thione group). The asterisk (*) is used herein to indicate the point at which a mono- or bivalent radical depicted is connected to the structure to which it relates and of which the radical forms part.

In a preferred embodiment of the pro-drugs of Amaryllidaceae isocarbostyril derivatives of the invention, R₃═H, OH, a linear O—C₁-C₂₄-alkyl, such as O-n-Methyl, O-n-Ethyl, O-n-Propyl, O-n-Butyl, O-n-Pentyl, O-n-Hexyl, O-n-Heptyl, O-n-Octyl, O-n-Nonyl, O-n-Decyl, O-n-Undecyl, O-n-Dodecyl, etc.

More preferably, R₃ is O—C₄H₉ as in Formula (IIe) and (IIf):

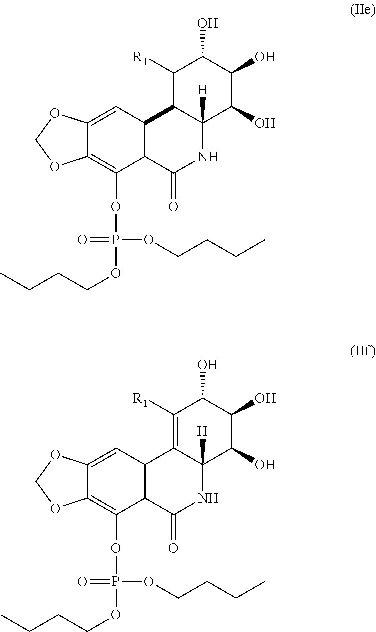

wherein
R₁ is selected from the group comprising: H, OH, O—C₁-C₆alkyl.

More preferably, R₃ is O-aryl, such as O-phenyl as in Formula (IIg) and (IIh):

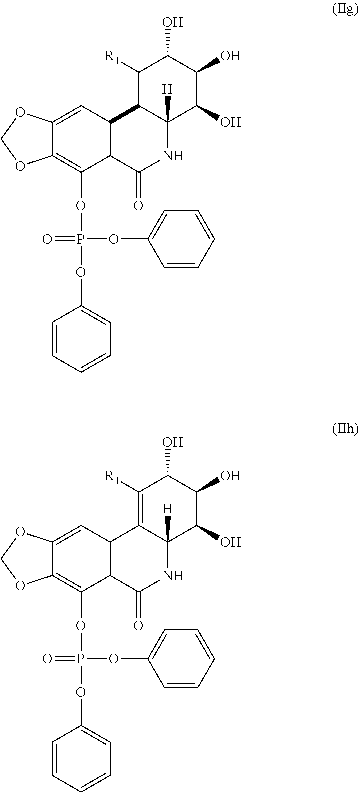

wherein
R₁ is selected from the group comprising: H, OH, O—C₁-C₆alkyl.

The invention particularly provides for the use of the pro-drugs of Amaryllidaceae isocarbostyril derivatives as identified by any of the formula's herein or the pharmaceutically acceptable addition salts, hydrates or solvates thereof and/or their stereoisomers, tautomers or diastereoisomers in the treatment of tumors located in the brain.

The invention particularly provides the pro-drugs of Amaryllidaceae isocarbostyril derivatives as identified herein or the pharmaceutically acceptable addition salts, hydrates or solvates thereof and/or their stereoisomers, tautomers or diastereoisomers, for the manufacturing of a medicament for treating tumors or cancers of the brain.

The invention further provides a pharmaceutical composition comprising a pharmaceutically acceptable excipient and a therapeutically effective amount of at least one of the pro-drugs of Amaryllidaceae isocarbostyril derivatives as identified herein or the pharmaceutically acceptable addition salts, hydrates or solvates thereof and/or their stereoisomers, tautomers or diastereoisomers.

The invention further provides for the use of one or more pro-drugs of Amaryllidaceae isocarbostyril derivatives as identified herein or the pharmaceutically acceptable addition salts, hydrates or solvates thereof and/or their stereoisomers, tautomers or diastereoisomers for the preparation of a medicament for the prevention and/or treatment of cancer, preferably of cancer in the CNS or brain.

The invention also provides a method for treating cancer, preferably CNS or brain cancer using a medicament comprising at least one pro-drug of Amaryllidaceae isocarbostyril derivatives as identified herein or the pharmaceutically acceptable addition salts, hydrates or solvates thereof and/or their stereoisomers, tautomers or diastereoisomers as an active ingredient, such that the cancer is treated.

The present invention further provides kits for use in treating cancer, preferably CNS cancer or brain cancer and related disorders in an individual in need thereof comprising a therapeutically effective amount of the pharmaceutical composition comprising at least one pro-drug of Amaryllidaceae isocarbostyril derivatives as identified herein or the pharmaceutically acceptable addition salts, hydrates or solvates thereof and/or their stereoisomers, tautomers or diastereoisomers, optionally, in combination with a pharmaceutically acceptable carrier.

Other aspects, embodiments, uses and advantages of the invention will become clear from the further description below.

### BRIEF DESCRIPTION OF THE DRAWINGS

FIG. 1: Summary of eEF1A roles in cell biology

FIG. 2: Potential binding sites for the Amaryllidaceae isocarbostyril derivatives on eEF1A protein: A: Three potential binding pockets of Narciclasine were found in the 3D structure of yeast eEF1A in independent docking experiments. They are shown here on the same structure. Pocket “a” corresponds to the GTP binding site. Pocket “c” is located in the binding region of the nucleotide exchange factor. The three eEF1A domains are colored in grey, red and green. The positions of residues lining each binding pocket are indicated as red spheres and their numbers are given. The insert shows the same view of eEF1A structure in association with the nucleotide exchange factor colored in blue. B: The molecular structures of various potential ligands are illustrated together with their theoretical affinity scores calculated for all compounds in each binding site.

FIG. 3: Binding assay of Narciclasine to eEF1A.

FIG. 4: genomic expression of eEF1A1 (A) and eEF1A2 (B) in human gliomas.

FIG. 5: anti-tumor effects of Narciclasine versus temozolomide in human primary brain tumor models. A and B: hematoxilin eosin staining of the brain tumors that developed into the brain of nude mice after intracranial graft of Hs683 and GL-19 human glioblastoma cells (NB: normal brain). Anti-tumor effects were evaluated in terms of animal survival in C and D. TMZ: temozolomide; schedule of administration is mentioned between brackets (“aXb” meaning “a” administrations per week during “b” weeks).

FIG. 6: anti-tumor effects of Narciclasine versus taxol in human NSCLC (non small cell lung carcinoma) models. The left panel illustrates the lungs of mice bearing lung tumor (tumor tissue is the white nodular one; pink part is the residual normal lung tissue) and hematoxilin eosin staining of the lung tumor (middle) and the brain metastasis that developed into the brain of nude mice after the graft of A549 human NSCLC cells into the lung (lower panel). Anti-tumor effects were evaluated in terms of animal survival when cells are grafted into the lungs (upper right graph) or directly into the brain (right lower panel). Taxol (dose: 20 mg/kg) and Narciclasine (dose: 1 mg/kg) were administered once per week during 5 consecutive weeks.

FIG. 7: anti-tumor effects of Narciclasine in human melanoma models. The left panel illustrates typical hematoxilin eosin staining of subcutaneous tumor and lung metastasis developed when VM-21 human melanoma cells are grafted subcutaneously in nude mice and their corresponding survival analysis. Narciclasine was administered per os while dacarbazine was administered intra-peritoneally. Doses and schedules appear on the graphs (ai X bw meaning “a” administrations per week during “b” weeks). The right panel illustrates typical hematoxilin eosin staining of human melanoma brain tumor developed into the brain when VM-48 human brain metastatic melanoma cells are grafted into the brain of nude mice and their corresponding survival analysis. Narciclasine and temozolomide (TMZ) were administered per os. Doses and schedules appear on the graphs (same legend than left panel).

FIG. 8: example of Narciclasine standard curve established with 8 samples of each Narciclasine concentration.

FIG. 9: the chemical structure of the silicium column used here is presented in the center.

### DETAILED DESCRIPTION OF THE PREFERRED EMBODIMENTS

The present invention stems from the unexpected observation that although administration of Narciclasine in mouse models does generally not result in effective anti-cancer treatment, it does result in a tumor-inhibitory effect when the tumor is located in the CNS or brain of nude mice. This observation, together with the identification of the eEF1A receptor of Narciclasine in brain tumor cells, lead to the idea of improving the physicochemical properties of Narciclasine-derivatives and others Amaryllidaceae isocarbostyril products for accumulating into the brain. This would result in an increase of the concentration of Narciclasine in the brain, which is high enough for effective treatment without having to initially administer a toxic or lethal dose of Narciclasine to the subject.

When assayed in vivo, Narciclasine and derivatives become toxic before displaying actual anticancer activity (Ingrassia L et al., J Med Chem 2009; 52:1100-14): while the safe chronical dose of Narciclasine is of 1 mg/kg in mice, 5 mg/kg dose leads to plasmatic concentrations of >50 nM (IC50 value) for few hours only (Ingrassia L et al., J Med Chem 2009; 52:1100-14). This could explain, at least partly, why we did not observed any in vivo improvement of the survival of any type of pre-clinical cancer models that do not develop into the brain (Ingrassia L et al., J Med Chem 2009; 52:1100-14). Indeed, against all odds, Narciclasine displayed marked and significant therapeutic benefits for any types of cancers (e.g. carcinoma, melanoma, glioma) when developed into the brain at doses that are non toxic. Moreover, Al compounds' effects on brain tumors have been obtained in apoptosis-sensitive as well as apoptosis-resistant pre-clinical models. This is of particular importance when considering that metastases (including brain metastases; i.e. secondary brain tumors) and gliomas (primary brain tumors) are intrinsically resistant to apoptosis as we reviewed it recently (Lefranc F et al., J Clin Oncol 2005; 23:2411-22).

Narciclasine controls plant growth and it was quite unexpected that a natural compound that control plant growth (e.g. Narciclasine) can also control brain tumor growth (whatever the brain tumor type, i.e. primary (glioma) versus secondary (metastases from melanomas and carcinomas among others)) at non-toxic dose, while keeping unaffected normal human cells. We postulate that eEF1A is the common target in plants and brain tumor cells when Narciclasine display its anti-growth properties.

The therapeutic benefits obtained with those compounds in tumors located into the brain could relate to eEF1A isoform expression deregulation but also, at least partly, to their accumulation into the brain. This observation also clearly indicates that Al compounds, including Narciclasine, are able to cross the blood brain barrier (BBB) even if it could not be expected regarding the molecular characteristics known to be involved in this passage (see below).

Indeed, an efficient blood-brain barrier (BBB) penetration is mandatory to obtain an efficient drug (Krogsgaard-Larsen P. Textbook of drug design and discovery; Taylor and Francis Ed.; New-York 2002, Third Edition, pp 130-131).

In addition, Narciclasine is associated with a narrow therapeutic index: it must be administered at non toxic doses in human patient (the equivalent of 1 mg/kg in mice) and must accumulate in the target organ, i.e. the brain until it will reach therapeutic effects against malignant brain tumors (of any type). Narciclasine is inefficient against non-proliferating brain tissues, i.e. neural tissues, while it blocks the proliferation of highly proliferating cancer cells.

The invention thus pertains to the enablement of i) facilitating the BBB passage by narciclasine and others Amaryllidaceae isocarbostyril prodrugs and ii) rendering the Narciclasine and others Amaryllidaceae isocarbostyril prodrugs as lipophilic as possible for their accumulation into the brain.

While most prior art documents aim to render Narciclasine and others Amaryllidaceae isocarbostyril prodrugs as hydrophilic as possible for systemic delivery, the present invention claims the opposite strategy, i.e. rendering these Narciclasine and others Amaryllidaceae isocarbostyril prodrugs as lipophilic as possible (while still able to cross the BBB) in order to minimize as much as possible their rapid elimination when administered to the patients and optimize their accumulation into the brain. In essence, the invention provides for the use of Amaryllidaceae isocarbostyril prodrug derivatives of the invention for the treatment of brain tumors only.

Studies have shown the following physico-chemical properties to be important for discovery and development of CNS (central nervous system) drugs because they are more restrictive at the BBB than at most other membrane barriers in the body (Kerns E H, L Li. Drug-like properties: concepts, structure and design and methods: from ADME to Toxicity optimization. Elsevier Inc. 2008, pp 130-131):
Hydrogen bonds (acceptors and donors)
Lipophilicity (log P)
Polar surface area (PSA)
Molecular Weight (MW)
Acidity.

Narciclasine and pancratistatin do not display good properties in term of hydrogen bonds, lipophilicity and polar surface area (PSA). Here, to improve the therapeutic potential of Al compounds for brain tumors (primary and secondary tumors), we choose to modify this class of molecules in order to decrease hydrogen bonds, polar surface area and mostly and consequently to increase their lipophilic properties. This is thus the opposite strategy to the approach and patent of GR Pettit et al (WO2004/052298 A2) who aimed to increase their hydrosolubility.

The invention pertains in essence to derivatives of Amaryllidaceae Isocarbostyril compounds having higher lipophilic properties than those known in the prior art, evaluated by means of the simple log P value.

Log P value is the partition coefficient (analytical chemistry) defined as follows: in the equilibrium distribution of a solute between two liquid phases (here a lipophilic one, e.g. 1-octanol, and an aqueous one, e.g. Phosphate Buffered Saline), the constant ratio of the solute's concentration in the upper phase to its concentration in the lower phase. The optimal log P value for penetration of the BBB is around 2.1 (Krogsgaard-Larsen P. Textbook of drug design and discovery; Taylor and Francis Ed.; New-York 2002, Third Edition, pp 130-131) and the calculated log P of Narciclasine and pancratistatin are weak (respectively average log P=−0.57 (+/−0.52) for Narciclasine and −1.27 (+/−0.52) for pancratistatin) (logP is calculated via http://www.vcclab.org/lab/alogps/start.html). Because modifications of these isocarbotyril derivatives implicate a loss of the biological activity (Ingrassia L et al., J Med Chem 2009; 52:1100-14), we choose here to modify prodrugs of these molecules. Cyclic phosphate prodrugs of these molecules are already known (see below (3)) from GR Pettit but physicochemical properties of these molecules make them poor candidates for crossing the BBB because log P values are around 0 (for (3a) it is of −1.25 (+/−1.45) (range=−2.60<log P<+0.20) (logP is calculated via http://www.vcclab.org/lab/alogps/start.html; Pettit G R and Melody N. J. Nat. Prod. 2005, 68, 207-211).

Known 3,4-cyclic phosphate prodrugs of natural isocarbostyril constituents are as follows:

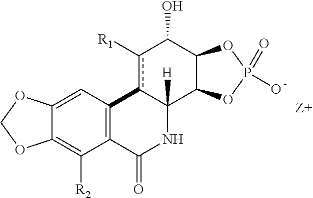

(3):
(3a): double bond with R₁═H, R₂═OH

### (3b): R₁═OH R₂═OH

(3c): R₁═H without double bond, R₂═OH
(3d): R₁═H without double bond, R₂═H
(3e): double bond with R₁═H, R₂═H

To this end, two aspects of the Narciclasine compound were taken into consideration:

3) the ability of crossing the blood-brain-barrier (BBB) and
4) the ability to accumulate in the brain, in order to achieve a local rise in the dose of Narciclasine in the brain, needed for achieving effective anti-tumor effects in the brain.

To this end, the pro-drugs of Amaryllidaceae isocarbostyril derivatives were developed that were more lipophillic, resulted in an increased retention into the brain, while retaining an acceptable crossing of the BBB.

The invention thus provides for pro-drugs of Amaryllidaceae isocarbostyril derivatives of the following general formula (I)

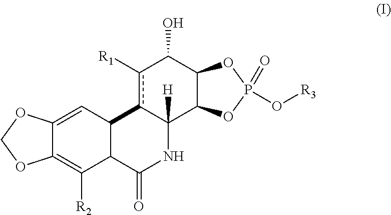

wherein the dotted line can be a single or double bond as exemplified by subgroups of general formulas (Ia) and (Ib):

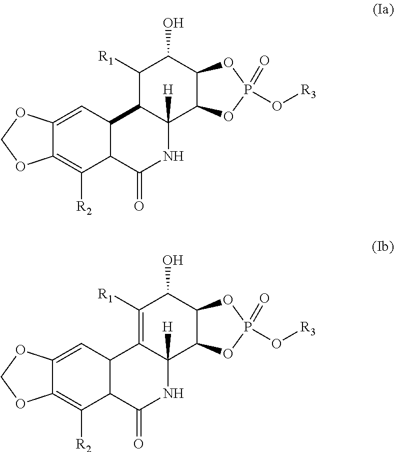

wherein in each of the formulas according to I, Ia or Ib:
R₁ is selected from the group comprising: H, OH, OR₄, NR₅R₆, alkyl, aryl, cycloC₁-C₆alkyl, cyclohexyl;
R₂ is selected from the group comprising: H, or OR₇;
R₃ is selected from the group comprising: a substituted or non-substituted straight or branched chain C₁-C₂₄alkyl group, a substituted or non-substituted aryl group, preferably substituted or non-substituted C₆-C₁₈aryl group, a substituted or non-substituted heteroaryl group, a substituted or non-substituted arylalkyl group, preferably a C₆-C₁₈arylC₁-C₆alkyl group, a cycloC₁-C₆alkyl group, a polyoxyalkylene chain of the formula (CxHyO)p, where x is 3 or more carbon atoms, p is 2 or more, and y is from 2x−2 to 2x, a heterocyclyl-C₁-C₂₄alkyl group or a heteroaryl-C₁-C₂₄alkyl, a —C₃-C₂₄alkynyl group, or a —C₃-C₂₄alkenyl group;
R₄, R₅, R₆ and R₇ are each independently selected from the group comprising: H, C₁-C₆alkyl, C₆-C₁₈aryl, carbonyl-C₁-C₆alkyl; carbonyl-C₆-C₁₀aryl;
each group optionally being substituted by a C₁-C₆-alkyl, OXO or ═S.

In a further embodiment, the invention provides pro-drugs of Amaryllidaceae isocarbostyril derivatives of the following general formula (Ic) or (Id):

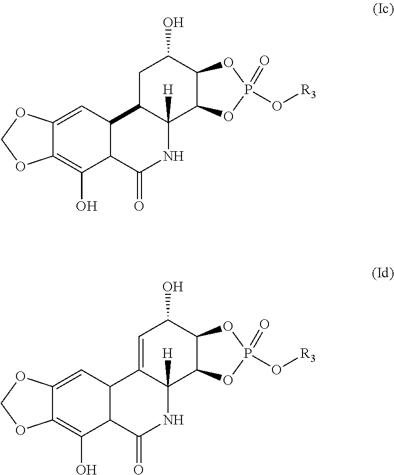

wherein
R₃ is selected from the group comprising: a substituted or non-substituted straight or branched chain C₁-C₂₄alkyl group, a substituted or non-substituted aryl group, preferably substituted or non-substituted C₆-C₁₈aryl group, a substituted or non-substituted heteroaryl group, a substituted or non-substituted arylalkyl group, preferably a C₆-C₁₈arylC₁-C₆alkyl group, a cycloC₁-C₆alkyl group, a polyoxyalkylene chain of the formula (CxHyO)p, where x is 3 or more carbon atoms, p is 2 or more, and y is from 2x−2 to 2x, a heterocyclyl-C₁-C₂₄alkyl group or a heteroaryl-C₁-C₂₄alkyl, a —C₃-C₂₄alkynyl group, or a —C₃-C₂₄alkenyl group;

In a preferred embodiment of the pro-drugs of Amaryllidaceae isocarbostyril derivatives of the invention, R₃=a linear C₁-C₂₄-alkyl, such as n-Methyl, n-Ethyl, n-Propyl, n-Butyl, n-Pentyl, n-Hexyl, n-Heptyl, n-Octyl, n-Nonyl, n-Decyl, n-Undecyl, n-Dodecyl, etc.

More preferably, R₃ is C₇H₁₅ as in Formula (Ie) and (If):

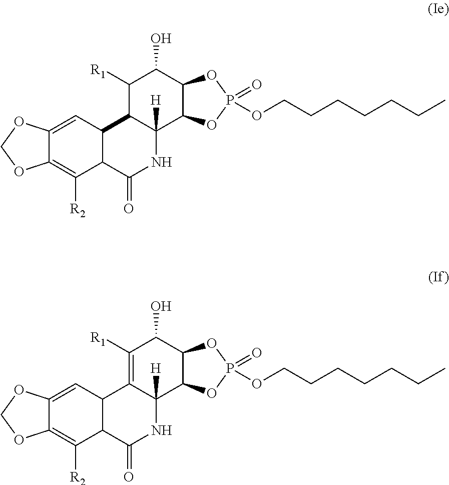

wherein
R₁ and R₂ are each independently selected from the group comprising: H, OH, O—C₁-C₆alkyl.

In a further embodiment, the invention provides pro-drugs of Amaryllidaceae isocarbostyril derivatives of the following general formula (Ig) or (Ih):

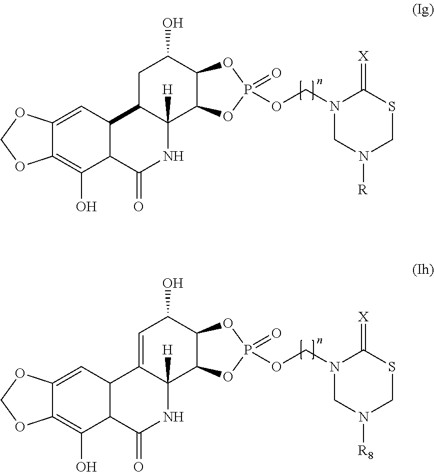

wherein
X is selected from O or S;
n is an integer selected from: 1, 2, 3, 4, 5, 6, 7, 8, 9, 10, 11, 12, 13, 14, 15, 16, 17, 18, 19, 20, 21, 22, 23, or 24.
R₈ is selected from the group comprising: H, C₁-C₆alkyl,

In a further preferred embodiment of the pro-drugs of Amaryllidaceae isocarbostyril derivatives of the invention is as in Formula (Ii) or (Ij):

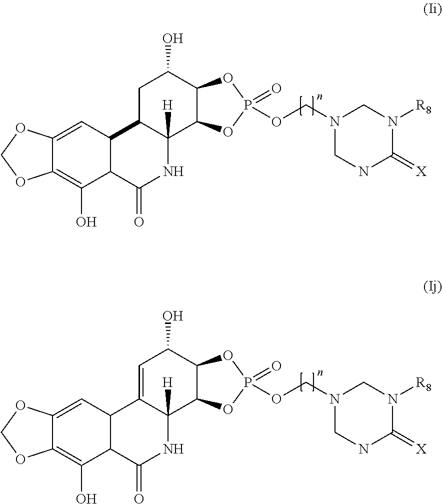

wherein
X is selected from O or S;
n is an integer selected from: 1, 2, 3, 4, 5, 6, 7, 8, 9, 10, 11, 12, 13, 14, 15, 16, 17, 18, 19, 20, 21, 22, 23, or 24.
R₈ is selected from the group comprising: H, C₁-C₆alkyl,

In a further preferred embodiment of the pro-drugs of Amaryllidaceae isocarbostyril derivatives of the invention is as in Formula (Ik) or (Il):

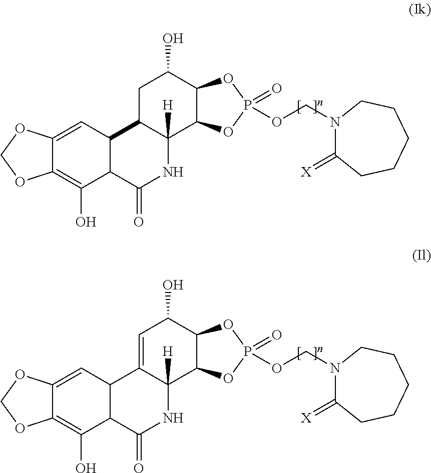

wherein
X is selected from O or S;
n is an integer selected from: 1, 2, 3, 4, 5, 6, 7, 8, 9, 10, 11, 12, 13, 14, 15, 16, 17, 18, 19, 20, 21, 22, 23, or 24.

The invention especially also provides pro-drugs of Amaryllidaceae isocarbostyril derivatives of the following general formula (II):

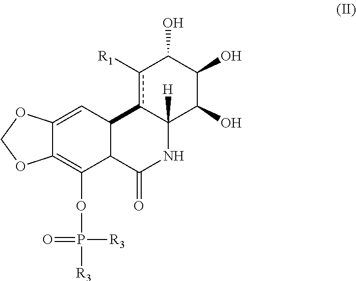

wherein the dotted line can be a single or double bond as exemplified by subgroups of general formulas (IIa) and (IIb):

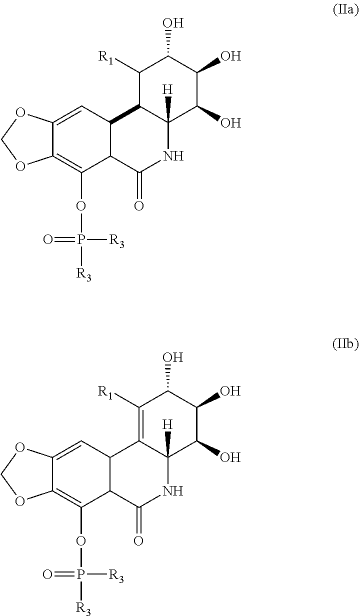

wherein in any one of the formulas II, IIa or IIb:
R₁ is selected from the group comprising: H, OH, OR₄, NR₅R₆, alkyl, aryl, cycloC₁-C₆alkyl, cyclohexyl;
R₃ is independently selected from the group comprising: H, OH, a substituted or non-substituted straight or branched chain O—C₁-C₂₄alkyl, a substituted or non-substituted O-aryl group, preferably substituted or non-substituted O—C₆-C₁₈aryl, a substituted or non-substituted O-heteroaryl group, a substituted or non-substituted O-aryl-alkyl group, preferably a O—C₆-C₁₈arylC₁-C₆alkyl group, a oxy-cycloC₁-C₆alkyl group, a O-polyoxyalkylene chain of the formula O—(CxHyO)p, where x is 3 or more carbon atoms, p is 2 or more, and y is from 2x−2 to 2x, a O-heterocyclyl-C₁-C₂₄alkyl group, a O-heteroaryl-C₁-C₂₄alkyl group, a O—C₃-C₂₄alkynyl group, or a O—C₃-C₂₄alkenyl group;
R₄, R₅, R₆ and R₇ are each independently selected from the group comprising: H, C₁-C₆alkyl, C₆-C₁₈aryl, carbonyl-C₁-C₆alkyl; carbonyl-C₆-C₁₀aryl;
each group optionally being substituted by a C₁-C₆-alkyl, OXO or ═S.

In a further embodiment, the invention provides pro-drugs of Amaryllidaceae isocarbostyril derivatives of the following general formula (IIc) or (IId):

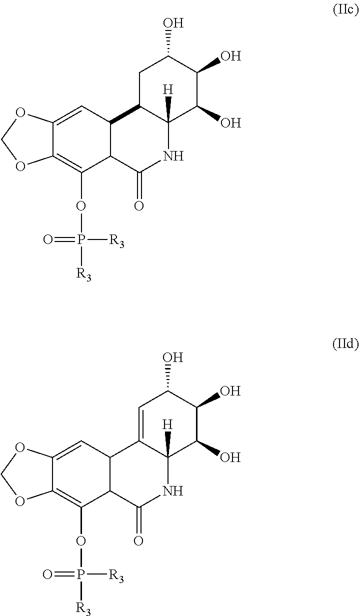

wherein
R₃ is independently selected from the group comprising: H, OH, a substituted or non-substituted straight or branched chain O—C₁-C₂₄alkyl, a substituted or non-substituted O-aryl group, preferably substituted or non-substituted O—C₆-C₁₈aryl, a substituted or non-substituted O-heteroaryl group, a substituted or non-substituted O-aryl-alkyl group, preferably a O—C₆-C₁₈arylC₁-C₆alkyl group, a oxy-cycloC₁-C₆alkyl group, a O-polyoxyalkylene chain of the formula O—(CxHyO)p, where x is 3 or more carbon atoms, p is 2 or more, and y is from 2x−2 to 2x, a O-heterocyclyl-C₁-C₂₄alkyl group, a O-heteroaryl-C₁-C₂₄alkyl group, a O—C₃-C₂₄alkynyl group, or a O—C₃-C₂₄alkenyl group;
each group optionally being substituted by a C₁-C₆-alkyl, *=O (OXO-group) or *=S (thione group). The asterisk (*) is used herein to indicate the point at which a mono- or bivalent radical depicted is connected to the structure to which it relates and of which the radical forms part.

In a preferred embodiment of the pro-drugs of Amaryllidaceae isocarbostyril derivatives of the invention, R₃═H, OH, a linear O—C₁-C₂₄-alkyl, such as O-Methyl, O-Ethyl, O-n-Propyl, O-n-Butyl, O-n-Pentyl, O-n-Hexyl, O-n-Heptyl, O-n-Octyl, O-n-Nonyl, O-n-Decyl, O-n-Undecyl, O-n-Dodecyl, etc.

More preferably, R₃ is O—C₄H₉ as in Formula (IIe) and (IIf):

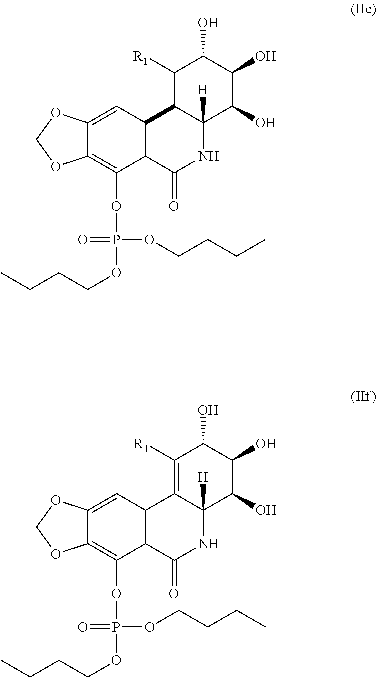

wherein
R₁ is selected from the group comprising: H, OH, O—C₁-C₆alkyl.

More preferably, R₃ is O-aryl as in Formula (IIg) and (IIh):

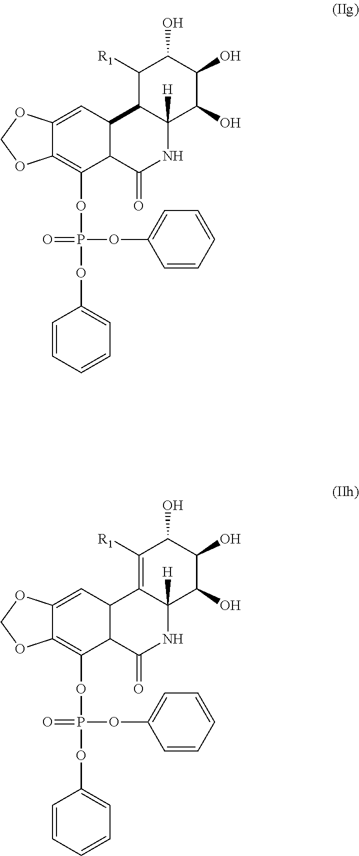

wherein
R₁ is selected from the group comprising: H, OH, O—C₁-C₆alkyl.

In a preferred embodiment of any of the formulas II, IIa-IIh described herein, R₁ is hydrogen.

When describing the compounds of the invention, the terms used are to be construed in accordance with the following definitions, unless a context dictates otherwise.

Whenever the term “substituted” is used in the present invention, it is meant to indicate that one or more hydrogens on the atom indicated in the expression using “substituted”, is replaced with a selection from the indicated groups, provided that the indicated atom's normal valency is not exceeded, and that the substitution results in a chemically stable compound, i.e. a compound that is sufficiently robust to survive isolation to a useful degree of purity from a reaction mixture, and formulation into a therapeutic agent.

The term “alkyl” by itself or as part of another substituent refers to a hydrocarbyl radical of Formula CₙH₂₊₁ wherein n is a number greater than or equal to 1. Alkyl groups may be linear or branched, and may be substituted as indicated herein. When a subscript is used herein following a carbon atom, the subscript refers to the number of carbon atoms that the named group may contain. Thus, for example, C₁₋₆alkyl includes all linear, or branched alkyl groups with between 1 and 6 carbon atoms, and thus includes such as for example methyl, ethyl, n-propyl, i-propyl, 2-methyl-ethyl, butyl and its isomers (e.g. n-butyl, i-butyl and tert-butyl); pentyl and its isomers, hexyl and its isomers, and the like. The term “C₁₋₆alkyl” as a group or part of a group refers to a hydrocarbyl radical of Formula CₙH₂₊₁ wherein n is a number ranging from 1 to 6. Generally, alkyl groups of this invention comprise from 1 to 6 carbon atoms, such as 1, 2, 3, 4, 5, or 6 carbon atoms, preferably from 1 to 4 carbon atoms, more preferably from 1 to 3 carbon atoms, still more preferably 1 to 2 carbon atoms. Examples of alkyl groups are methyl, ethyl, n-propyl, 1-methylethyl, i-propyl, butyl and its isomers (e.g. n-butyl, i-butyl and t-butyl), 1-methylpropyl, 2-methylpropyl, 1,1-dimethylethyl, n-pentyl and its isomers, 1-methylbutyl, 2-methylbutyl, 3-methylbutyl, 2,2-dimethylpropyl, 1-ethylpropyl, n-hexyl and its isomers, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethyl butyl, 3,3-dimethyl butyl, 1-ethyl butyl, 2-ethyl butyl, 1,1,2-trimethylpropyl, 1,2,2-trimethylpropyl, 1-ethyl-1-methylpropyl, 1-ethyl-2-methylpropyl, heptyl and its isomers, octyl and its isomers, nonyl and its isomers, decyl and its isomers etc. Exemplary alkyl chains or groups are those occurring in saturated fatty acids such as: Lauric acid (12 C) Myristic acid (14 C) Palmitic acid (16 C) Stearic acid (18 C) Arachidic acid (20 C) etc.

The term “heteroaryl” as a group or part of a group is selected from pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, purinyl, imidazo[1,2-a]pyridinyl.

The term “cycloalkyl” as used herein is a cyclic alkyl group, a monovalent, saturated, or unsaturated hydrocarbyl group having 1, 2, or 3 cyclic structures. Cycloalkyl includes all saturated or partially saturated (containing 1 or 2 double bonds) hydrocarbon groups containing 1 to 3 rings, including monocyclic, bicyclic, or polycyclic alkyl groups. Cycloalkyl groups may comprise 3 or more carbon atoms in the ring and generally, according to this invention comprise from 3 to 10, more preferably from 3 to 8 carbon atoms still more preferably from 3 to 6 carbon atoms. The further rings of multi-ring cycloalkyls may be either fused, bridged, and/or joined through one or more spiro atoms. Examples of cycloalkyl groups are: cyclopropyl, cyclobutyl, cyclopentyl, cyclohexyl, cycloheptyl, cyclooctyl, cyclononyl, and cyclodecyl.

The term “alkenyl” as used herein refers to an unsaturated hydrocarbyl group, which may be linear, branched, or cyclic, comprising one or more carbon-carbon double bonds. Alkenyl groups thus comprise two or more carbon atoms, preferably between 2 and 24 carbon atoms. Non-limiting examples are propenyl, 2-methylpropenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl, and the like. Examples of preferred alkenyl groups are those that occur in unsaturated fatty acids, such as e.g. Myristoleic acid, Palmitoleic acid, Sapienic acid, Oleic acid, Linoleic acid, α-Linolenic acid, Arachidonic acid, Eicosapentaenoic acid, Erucic acid, Docosahexaenoic acid.

The term “alkynyl” as used herein, similarly to alkenyl, refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkenyl groups. Examples alkynyl groups are ethynyl, 2-propynyl, 2-butynyl, 3-butynyl, 2-pentynyl and its isomers, 2-hexynyl and its isomers, 2-heptynyl and its isomers, 2-octynyl and its isomers, and the like.

The term “heterocyclyl” alone or in combination, is defined as a saturated or partially unsaturated monocyclic, bicyclic, or polycyclic heterocycle having preferably 3 to 12 ring members, more preferably 5 to 10 ring members, and more preferably 5 to 6 ring members, which contains one or more heteroatom ring members selected from nitrogen, oxygen, or sulfur, and which is optionally substituted on one or more carbon atoms by alkyl, alkoxy, halogen, hydroxyl, oxo, optionally mono- or disubstituted amino, nitro, cyano, haloalkyl, carboxyl, alkoxycarbonyl, cycloalkyl, optionally mono- or disubstituted aminocarbonyl, methylthio, methylsulfonyl, aryl, and a saturated or partially unsaturated monocyclic, bicyclic or tricyclic heterocycle having 3 to 12 ring members which contains one or more heteroatom ring members selected from nitrogen, oxygen, or sulfur, and whereby the optional substituents on any amino function are independently selected from alkyl, alkoxy, aryl, aryloxy, aryloxyalkyl, aralkyl, alkyloxycarbonylamino, amino, and aminoalkyl whereby each of the amino groups may optionally be mono- or where possible di-substituted with alkyl. Exemplary heterocyclic groups include aziridinyl, oxiranyl, thiiranyl, piperidinyl, azetidinyl, imidazolinyl, imidazolidinyl, pyrazolidinyl, isoxazolinyl, oxazolidinyl, isoxazolidinyl, thiazolidinyl, isothiazolidinyl, piperidinyl, succinimidyl, 3H-indolyl, indolinyl, isoindolinyl, chromenyl, isochromanyl, xanthenyl, 2H-pyrrolyl, 1-pyrrolinyl, 2-pyrrolinyl, 3-pyrrolinyl, pyrrolidinyl, 4H-quinolizinyl, 4aH-carbazolyl, 2-oxopiperazinyl, piperazinyl, homopiperazinyl, 2-pyrazolinyl, 3-pyrazolinyl, pyranyl, dihydro-2H-pyranyl, 4H-pyranyl, 3,4-dihydro-2H-pyranyl, oxetanyl, thietanyl, 3-dioxolanyl, 1,4-dioxanyl, 2,5-dioximidazolidinyl, 2,2,4-piperidonyl, 2-oxopiperidinyl, 2-oxopyrrolodinyl, 2-oxoazepinyl, indolinyl, tetrahydropyranyl, tetrahydrofuranyl, tetrehydrothienyl, tetrahydroquinolinyl, tetrahydroisoquinolinyl, thiomorpholinyl, thiomorpholinyl sulfoxide, thiomorpholinyl sulfone, 1,3-dioxolanyl, 1,4-oxathianyl, 1,4-dithianyl, 1,3,5-trioxanyl, 6H-1,2,5-thiadiazinyl, 2H-1,5,2-dithiazinyl, 2H-oxocinyl, 1H-pyrrolizinyl, tetrahydro-1,1-dioxothienyl, N-formylpiperazinyl, and morpholinyl.

The term “aryl” as used herein refers to a polyunsaturated, aromatic hydrocarbyl group having a single ring (i.e. phenyl) or multiple aromatic rings fused together (e.g. naphthalene or anthracene), or linked covalently, typically containing 6 to 18 atoms; wherein at least one ring is aromatic. The aromatic ring may optionally include one to three additional rings (either cycloalkyl, heterocyclyl, or heteroaryl) fused thereto. Aryl is also intended to include the partially hydrogenated derivatives of the carbocyclic systems enumerated herein. Non-limiting examples of aryl comprise phenyl, biphenylyl, biphenylenyl, 5- or 6-tetralinyl, 1-, 2-, 3-, 4-, 5-, 6-, 7-, or 8-azulenyl, 1- or 2-naphthyl, 1-, 2-, or 3-indenyl, 1-, 2-, or 9-anthryl, 1- 2-, 3-, 4-, or 5-acenaphtylenyl, 3-, 4-, or 5-acenaphtenyl, 1-, 2-, 3-, 4-, or 10-phenanthryl, 1- or 2-pentalenyl, 1,2-, 3-, or 4-fluorenyl, 4- or 5-indanyl, 5-, 6-, 7-, or 8-tetrahydronaphthyl, 1,2,3,4-tetrahydronaphthyl, 1,4-dihydronaphthyl, dibenzo[a,d]cylcoheptenyl, 1-, 2-, 3-, 4-, or 5-pyrenyl. The aryl ring can optionally be substituted by one or more substituents.

The terms “stereoisomers”, “tautomers” and “diastereoisomers” have their general meaning in the art. Stereoisomers are isomeric molecules that have the same molecular formula and sequence of bonded atoms and differ only in the three-dimensional orientations of their atoms in space. In chemistry, an enantiomer is one of two stereoisomers that are mirror images of each other that are “non-superposable” (not identical), much as one's left and right hands are “the same” but opposite. Two compounds that are enantiomers of each other have the same physical properties, except for the direction in which they rotate polarized light and how they interact with different optical isomers of other compounds. The mixture of both left and right rotating enantiomers in an equal amount is called the racemate or racemic mixture. Diastereomers or diastereoisomers are stereoisomers not related through a reflection operation. They are not mirror images of each other. These include meso compounds, cis-trans (E-Z) isomers, and non-enantiomeric optical isomers. Diastereomerism occurs when two or more stereoisomers of a compound have different configurations at one or more (but not all) of the equivalent (related) stereocenters and are not mirror images of each other. When two diastereoisomers differ from each other at only one stereocenter they are epimers. Each stereocenter gives rise to two different configurations and thus to two different stereoisomers. Diastereomers differ from enantiomers in that the latter are pairs of stereoisomers which differ in all stereocenters and are mirror images of each other. Tautomers are isomers of organic compounds that readily interconvert by a chemical reaction called tautomerization. It is common that this reaction results in the formal migration of a hydrogen atom or proton, accompanied by a switch of a single bond and adjacent double bond. In solutions in which tautomerization is possible, a chemical equilibrium of the tautomers will be reached. The exact ratio of the tautomers depends on several factors, including temperature, solvent, and pH.

As used in the specification and the appended claims, the singular forms “a”, “an,” and “the” include plural referents unless the context clearly dictates otherwise. By way of example, “a compound” means one compound or more than one compound.

Whenever used in the present invention the term “compounds of the invention” or “pro-drugs of Amaryllidaceae isocarbostyril derivatives of the invention” or a similar term is meant to include the compounds as displayed in the formulas defined herein and any subgroup thereof. This term also refers to the compounds as depicted in Table A and their derivatives, salts, solvates, hydrates, esters, and metabolites.

TABLE A

| | General | | | |
|---|---|---|---|---|
| ID | Formula | R₁ | R₂ | R₃ |
| N1 | Ia | H | OH | C₇H₁₅ |
| N2 | Ib | H | OH | C₇H₁₅ |
| N3 | Ia | H | OH | Phenyl |
| N4 | Ib | H | OH | Phenyl |
| | General | | | |
| ID | Formula | X | R₈ | n |
| N5 | Ig | S | CH₃ | 1 |
| N6 | Ih | S | CH₃ | 1 |
| N7 | Ig | S | CH₃ | 2 |
| N8 | Ih | S | CH₃ | 2 |
| N9 | Ig | S | CH₃ | 3 |
| N10 | Ih | S | CH₃ | 3 |
| N11 | Ig | S | CH₃ | 4 |
| N12 | Ih | S | CH₃ | 4 |
| N13 | Ii | S | CH₃ | 1 |
| N14 | Ij | S | CH₃ | 1 |
| N15 | Ii | S | CH₃ | 2 |
| N16 | Ij | S | CH₃ | 2 |
| N17 | Ii | S | CH₃ | 3 |
| N18 | Ij | S | CH₃ | 3 |
| N19 | Ii | S | CH₃ | 4 |
| N20 | Ij | S | CH₃ | 4 |
| N21 | Ik | O | | 1 |
| N22 | Il | O | | 1 |
| N23 | Ik | O | | 2 |
| N24 | Il | O | | 2 |
| N25 | Ik | O | | 3 |
| N26 | Il | O | | 3 |
| N27 | Ik | O | | 4 |
| N28 | Il | O | | 4 |
| | General | | | |
| ID | Formula | R₁ | R₂ | R₃ |
| N29 | Ia | H | OH | 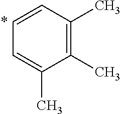 |
| N30 | Ib | H | OH | 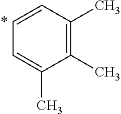 |
| N31 | IIc | H | OC₄H₉ | OC₄H₉ |
| N32 | IId | H | OC₄H₉ | OC₄H₉ |
| N33 | IIc | H | O-phenyl | O-phenyl |
| N34 | IId | H | O-phenyl | O-phenyl |

The pharmaceutically acceptable acid and base addition salts as mentioned hereinabove or hereinafter are meant to comprise the therapeutically active non-toxic acid and base addition salt forms which the compounds of the invention are able to form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, malonic, succinic (i.e. butanedioic acid), maleic, fumaric, malic, mandelic, tartaric, citric, methanesulfonic, muconic, 2-naphtalenesulfonic, ethanesulfonic, benzenesulfonic, benzoic, camphorsulfonic, glucoheptonic, gluconic, glutamic, glycolic, hydroxynaphtoic, 2-hydroxyethanesulfonic, p-toluenesulfonic, cyclamic, salicylic, p-aminosalicylic, pamoic, dibenzoyl-L-tartaric, trimethylsulfonic, trifluoroacetic acids and the like. Conversely said salt forms can be converted by treatment with an appropriate base into the free base form.

The compounds of the invention containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term solvate comprises the hydrates and solvent addition forms which the compounds of the invention are able to form, as well as the salts thereof. Examples of such forms are e.g. hydrates, alcoholates and the like.

The pro-drugs of Amaryllidaceae isocarbostyril derivatives of the invention contain one or more asymmetric carbon atoms that serve as a chiral center, which may lead to different optical forms (e.g. enantiomers or diastereoisomers). The invention comprises all such optical forms in all possible configurations, as well as mixtures thereof. More generally, from the above, it will be clear to the skilled person that the compounds of the invention may exist in the form of different isomers and/or tautomers, including but not limited to geometrical isomers, conformational isomers, E/Z-isomers, stereochemical isomers (i.e. enantiomers and diastereoisomers) and isomers that correspond to the presence of the same substituents on different positions of the rings present in the compounds of the invention. All such possible isomers, tautomers, and mixtures thereof are included within the scope of the invention.

The term “pro-drug” as used herein means the pharmacologically acceptable derivatives, preferably phosphates, such that the in vivo biotransformation product of the derivative is the active drug. The reference by Goodman and Gilman (The Pharmacological Basis of Therapeutics, 8th Ed, McGraw-Hill, Int. Ed. 1992, “Biotransformation of Drugs”, p 13-15) describing pro-drugs generally is hereby incorporated. Pro-drugs are characterized by increased bio-availability and are readily metabolized into the active drugs in vivo. A narciclasine pro-drug is known in the art by e.g. the group of Pettit et al., WO2004/052298 A2), disclosing phosphate pro-drug of Narciclasine, called narcistatin.

The compounds of the invention may be prepared using methods and chemistries with which those skilled in the art shall be familiar. Exemplary methods are described in the experimental section below.

The terms described above and others used in the specification are well understood to those in the art.

The compounds of the invention are particularly advantageous since while being anti-proliferative and anti-migratory for cancer cells, the compounds according to the invention exhibit a low toxicity level on healthy cells. The terms “toxicity” or “toxic effects” as used herein refer to the detrimental effect(s) a compound may have on healthy cells, tissues, or organs. The toxicity level of the compounds according to the invention is surprisingly low. The compounds according to the invention combine the essential features of a good anti-cancer activity and a low level of toxicity.

Consequently the compounds according to the invention may be used in pharmaceutical compositions for the treatment of various cancers. In addition, because they have a low level of toxicity the compounds according to the invention may be used during longer periods of treatments.

Due to these interesting properties, in particular the antiproliferative properties and the low level of toxicity, the compounds according to the invention are particularly suitable for use as a medicament, preferably in the treatment of cancer. Therefore, in another embodiment, the invention relates to compounds according to the invention for use as a medicament. The invention also encompasses the use of at least one compound of formula I or II as defined above, for the preparation of a medicament for treating cancer.

In another embodiment, the present invention relates to a pharmaceutical composition comprising a pharmaceutically acceptable excipient and a therapeutic amount of at least one compound according to the invention. The invention also encompasses the use of a pharmaceutical composition comprising a pharmaceutically acceptable excipient and a therapeutic amount of at least one compound according to the invention, for the preparation of a medicament for treating cancer.

The term “therapeutically effective amount” or “effective amount” as used herein refers to the quantity of compound or pharmaceutical composition that elicits the biological or medicinal response in a tissue, system, animal, or human that is being sought by a researcher, veterinarian, medical doctor or other clinician, which includes alleviation of the symptoms of the cancer being treated. In particular, these terms refer to the quantity of compound or pharmaceutical composition according to the invention which is necessary to prevent, cure, ameliorate, or at least minimize the clinical impairment, symptoms, or complications associated with cancer in either a single or multiple doses.

The pharmaceutical composition can be prepared in a manner known per se to one of skill in the art. For this purpose, at least one compound having formula I or II, one or more solid or liquid pharmaceutical excipients and, if desired, in combination with other pharmaceutical active compounds, are brought into a suitable administration form or dosage form which can then be used as a pharmaceutical in human medicine or veterinary medicine.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying, and suspending agents, dispersing agents, desintegrants, bulking agents, fillers, preserving agents, sweetening agents, flavoring agents, flow regulators, release agents, etc. The compositions may also be formulated so as to provide rapid, sustained, or delayed release of the active compound(s) contained therein, for example using liposomes or hydrophilic polymeric matrices based on natural gels or synthetic polymers.

Although the antitumor agent of the present invention may be composed of only the chemical pro-drug according to any one of the embodiments reflected herein, it is normally formulated in accordance with ordinary methods with one more types of pharmaceutically acceptable carriers and/or additives.

Examples of pharmaceutically acceptable carriers include water, pharmaceutically acceptable organic solvents, collagen, polyvinyl alcohol, polyvinyl pyrrolidone, carboxyvinyl polymers, sodium alginate, water-soluble dextran, sodium carboxymethyl starch, pectin, xanthan gum, gum Arabic, casein, gelatin, agar, glycerin, propylene glycol, polyethylene glycol, vaseline, paraffin, stearyl alcohol, stearic acid, human serum albumin, mannitol, sorbitol and lactose.

Examples of additives used during formulation include vehicles, disintegration agents, correctives, fillers, thickeners, binders, moisturizers, surface activators, lubricants, stabilizers, antimicrobials, buffers, isotonic agents, chelating agents, pH adjusters and surfactants, and these additives are suitably selected according to the form of the administration units of the preparation and so on.

Examples of administration routes include oral administration and parenteral administration such as intracerebral administration, intraperitoneal administration, intraoral administration, intratracheal administration, rectal administration, subcutaneous administration, intramuscular administration and intravenous administration. In addition, examples of administration forms include tablets, powders, injections, granules, sprays, capsules, syrups, emulsions, suppositories, ointments and tapes.

Although dosage and the number of administrations may generally be determined on an individual basis in consideration of the age, symptoms, body weight and administration effects of the patient or animal being dosed under the strict supervision of a supervising physician or veterinarian, in the case of a human adult, the general reference for the daily dosage in the case of venous administration is 1 mg/kg, and this daily dosage can be administered all at once or divided among several administrations per day.

The compounds of the invention may be especially used in (the preparation of a medicament for) the treatment of cancers such as but not limited to: all types of tumors, located in the CNS, including primary brain tumors such as astrocytoma, pilocytic astrocytoma, diffuse astrocytoma, anaplastic (malignant) astrocytoma, glioma, oligodendroglioma, mixed glioma such as oligoastrocytoma, choroid plexus papilloma, ependymoma, glioblastoma multiforme, medulloblastoma, acoustic neuroma, lymphoma, meningioma, pineal gland tumor, pituitary adenoma, schwannoma, etc., as well as secondary brain tumors, spreading or metastasizing into the brain such as: melanoma, breast cancer, renal cell carcinoma, colorectal cancer, etc.

Accordingly, the present invention provides a method for the treatment of brain cancer comprising administering to an individual an effective amount of at least one Amaryllidaceae isocarbostyril pro-drug derivative of the invention as an active ingredient, such that the cancer is treated. By way of example, in an embodiment of the invention, cancer is treated in a subject in need of treatment by administering to the subject a therapeutically effective amount of at least one pro-drug of Amaryllidaceae isocarbostyril derivative of the invention, effective to treat the cancer. The subject is preferably a mammal (e.g., humans, domestic animals, and commercial animals, including cows, dogs, monkeys, mice, pigs, and rats), and is most preferably a human.

The term “pharmaceutical composition” encompasses all formulations including the pro-drug of Amaryllidaceae isocarbostyril derivatives according to the invention as (one of) the active ingredient(s), formulated with a pharmaceutically acceptable carrier.

For these purposes, the pro-drugs of Amaryllidaceae isocarbostyril derivatives or the pharmaceutical compositions of the present invention may be administered in dosage unit formulations containing conventional non-toxic pharmaceutically acceptable carriers, adjuvants, and vehicles. The at least one pro-drug of Amaryllidaceae isocarbostyril derivatives or the pharmaceutical compositions of the invention will generally be administered in an “effective amount”, by which is meant any amount of at least one pro-drug of Amaryllidaceae isocarbostyril derivatives that, upon suitable administration, is sufficient to achieve the desired therapeutic or prophylactic effect in the individual to which it is administered. Usually, depending on the condition to be prevented or treated and the route of administration, such an effective amount will usually be between 0.01 to 1000 mg per kilogram body weight, more often between 0.1 and 500 mg, such as between 1 and 250 mg, for example about 5, 10, 20, 50, 100, 150, 200, or 250 mg, per kilogram body weight day of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender, weight and general condition of the patient and the nature and severity of the disease/symptoms to be treated. Reference is made to standard handbooks, such as the latest edition of Remington's Pharmaceutical Sciences.

In accordance with the method of the present invention, said pharmaceutical composition can be administered separately at different times during the course of therapy or concurrently in divided or single combination forms. The present invention is therefore to be understood as embracing all such regimes of simultaneous or alternating treatment and the term “administering” is to be interpreted accordingly.

Essentially, the primary modes of treatment of solid tumor cancers comprise surgery, radiation therapy, and chemotherapy, separately and in combination. The compounds according to the invention are suitable for use in combination with these medicinal techniques. The compounds of the invention may be useful in increasing the sensitivity of tumor cells to radiation in radiotherapy and also in potentiating or enhancing damage to tumors by chemotherapeutic agents. The compounds and their pharmaceutically acceptable salts and/or solvates may also be useful for sensitizing multidrug-resistant tumor cells. The compounds according to the invention are useful therapeutic compounds for administration in conjunction with DNA-damaging cytotoxic drugs or radiation used in radiotherapy to potentiate their effect.

In another embodiment of the method of the invention, the administration may be performed with food, e.g., a high-fat meal. The term “with food” means the consumption of a meal either during or no more than about one hour before or after administration of a pharmaceutical composition according to the invention.

For an oral administration form, the compositions of the present invention can be mixed with suitable additives, such as excipients, stabilizers, or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate, and lactose and/or other excipients, binders, extenders, disintegrants, diluents, and lubricants known in the art.

The oral administration of a pharmaceutical composition comprising at least one compound according to the invention, or a pharmaceutically acceptable salt or ester and/or solvate thereof, is suitably accomplished by uniformly and intimately blending together a suitable amount of said compound in the form of a powder, optionally also including a finely divided solid carrier, and encapsulating the blend in, for example, a hard gelatin capsule. The solid carrier can include one or more substances, which act as binders, lubricants, disintegrating agents, coloring agents, and the like. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Oral administration of a pharmaceutical composition comprising at least one compound according to the invention, or a pharmaceutically acceptable salt or ester and/or solvate thereof can also be accomplished by preparing capsules or tablets containing the desired amount of said compound, optionally blended with a solid carrier as described above. Compressed tablets containing the pharmaceutical composition of the invention can be prepared by uniformly and intimately mixing the active ingredient with a solid carrier such as described above to provide a mixture having the necessary compression properties, and then compacting the mixture in a suitable machine to the shape and size desired. Molded tablets maybe made by molding in a suitable machine, a mixture of powdered compound moistened with an inert liquid diluent.

When administered by nasal aerosol or inhalation, these compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions, or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant.

For subcutaneous or intravenous administration, the compound of the invention, if desired with the substances customary therefor such as solubilizers, emulsifiers, or further auxiliaries, are brought into solution, suspension, or emulsion. The compounds of the invention can also be lyophilized and the lyophilizates obtained used, for example, for the production of injection or infusion preparations. Suitable solvents are, for example, water, physiological saline solution, or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents, or solvents, such as mannitol, 1,3-butanediol, water, Ringer's solution, or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these formulations may be prepared by mixing the compounds according to the invention with a suitable non-irritating excipient, such as cocoa butter, synthetic glyceride esters, or polyethylene glycols, which are solid at ordinary temperatures, but liquidify and/or dissolve in the rectal cavity to release the drug.

The pharmaceutical compositions of this invention can be administered to humans in dosage ranges specific for each compound comprised in said compositions. The compounds comprised in said composition can be administered together or separately.

It will be understood, however, that specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In essence, the type of tumor treated by the pro-drugs of the invention is not decisive, apart from its localization in the brain. Narciclasine anti-tumor effects have been shown on all types of tumors only when located in the CNS. Primary brain tumors can be classified as follows: astrocytoma, the most common type of brain tumor in children, which originates in the brainstem, cerebellum, white matter of the cerebrum, or spinal cord; brainstem glioma, which originates in the medulla, pons, or midbrain; choroid plexus papilloma, which originates in the ventricles; ependymoma, which originates in the membrane that lines the ventricles and central canal of the spine; glioblastoma multiforme, the most common type in adults, which originates from glial cells in the cerebrum; medulloblastoma, the second most common type in children, which originates in the fourth cerebral ventricle and the cerebellum; often invades the meninges. Other types of primary brain cancer include the following: acoustic neurinoma, which originates in the vestibule-cochlear nerve; lymphoma, which originates from lymphocytes, common e.g. in HIV/AIDS patients; meningioma, which originates from the meninges; pineal gland tumor, a rare tumor which originates in the pineal gland; pituitary adenoma, which originates in surface cells of the pituitary gland, schwannoma, which originates in cells of the myelin sheath that covers neurons.

Secondary (metastatic) brain tumors are tumors that spread to the brain and can be grouped as follows: melanoma, breast cancer, renal cell carcinoma, colorectal cancer, bladder carcinoma, lung cancers, prostate cancers, lymphomas, sarcomas, among others.

The present invention also encompasses processes for the preparation of compounds of the invention and subgroups thereof as described in the examples section.

It can be necessary to protect reactive functional groups, for example hydroxy, amino, or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1999.

The invention furthermore enables to screen for anti-cancer agents that can specifically target brain tumor cells, based on the expression of the eEF1A receptor. This receptor exists in two distinct isoforms, i.e. eEF1A1 and eEF1A2 that are differentially expressed in brain cancer cells when compared to normal brain cells. The invention includes the testing of any type of compounds that can target one or the two eEF1A1 isoforms using cell models displaying one of the two eEF1A isoforms, or the two isoforms together. Biochemical readouts include analyses of protein syntheses and cell biology readouts include cell proliferation, cell death and cell migration analyses.

The following examples are meant to illustrate the present invention. These examples are presented to exemplify the invention and are not to be considered as limiting the scope of the invention.

### EXAMPLES

The invention is illustrated by the following non-limiting examples

### Example 1

### eEF1A as a Potential Target of Amaryllidaceae Isocarbostyril Derivatives in Brain Tumors

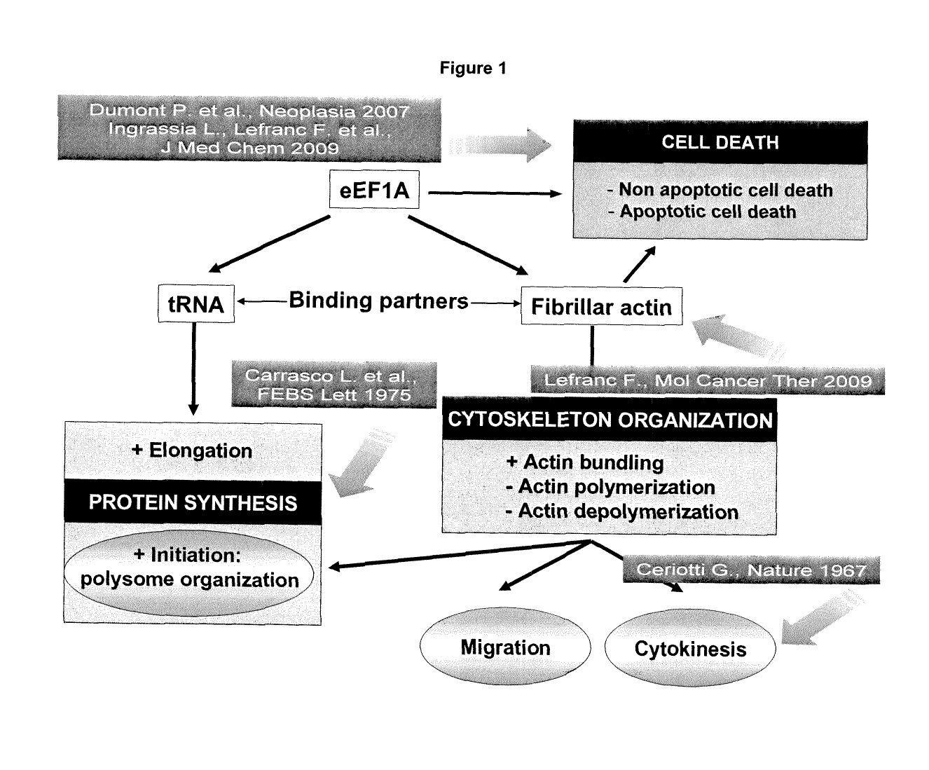

In this experiment, the inventors show that eEF1A is a target for the Al compounds at their IC₅₀ values, reconciling their effects on protein synthesis, actin cytoskeleton and cell death (FIG. 1), and that eEF1A-targeting represents a useful strategy to combat brain cancers.

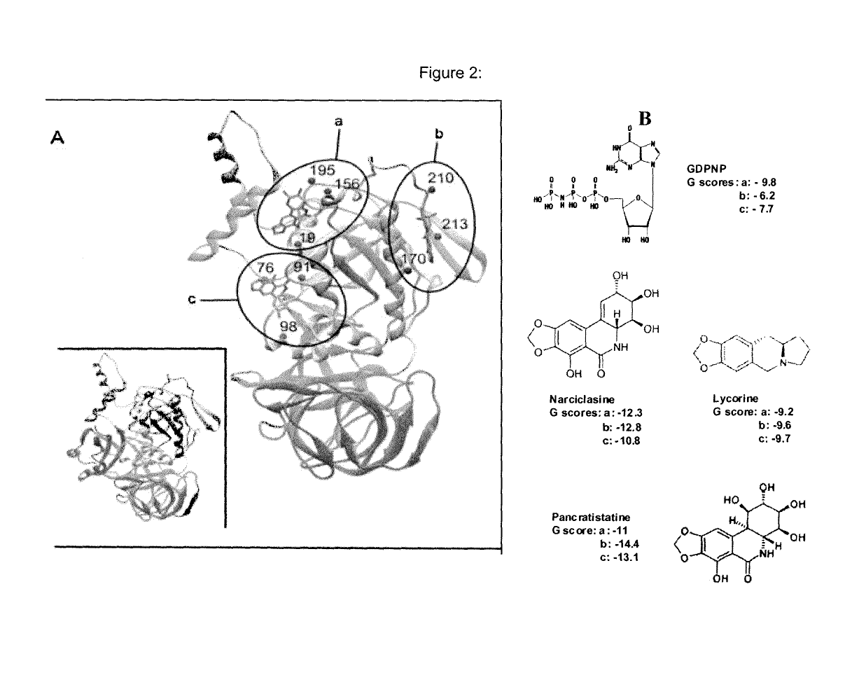

Docking experiments that were performed revealed that the Al's are potential eEF1A ligands. Indeed, three binding pockets in independent docking experiments conducted with the crystallographic structure of the yeast eEF1A (PDB code: 1G7C; human isoforms are not available) were found (FIG. 2A). One of the pockets corresponds to the GTP binding site (pocket “a”) and another to the binding region of the nucleotide exchange nucleotide exchange factor (pocket “c”). The molecular structures of the docked Al are presented in FIG. 2, together with their binding free energy score in each pocket. Narciclasine features affinity scores ranging between −12.3 to −10.8 kcal/mol. For comparison, the GDPNP, an analog of the natural ligand GTP, has a free energy score of −9.8 kcal/mol (FIG. 2B), thus weaker than the scores displayed by Narciclasine.

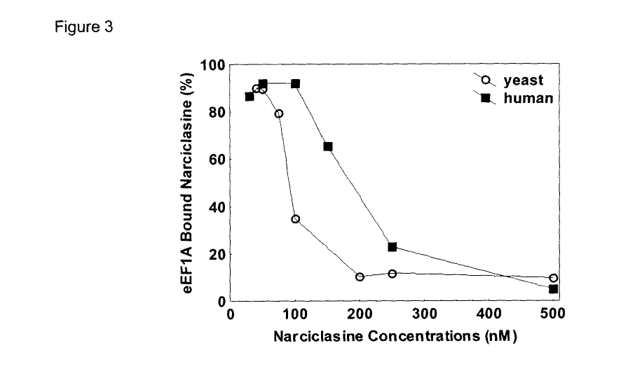

Binding assays confirmed that Al's are ligands for eEF1A proteins in tube assay and in cell cultures (FIG. 3).

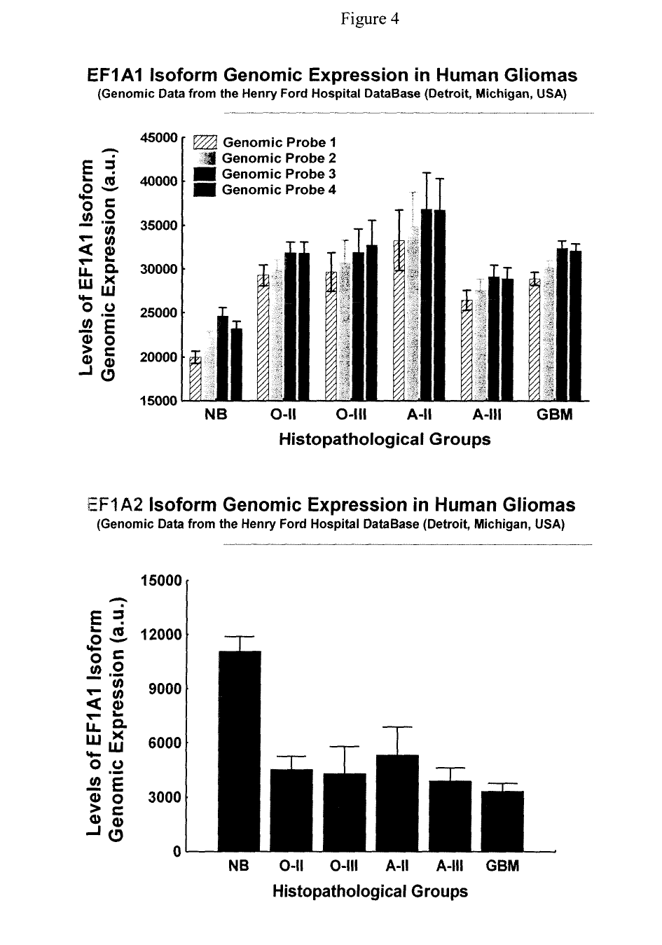

When looking at the expression levels of eEF1A isoforms in brain tumors, analyzes revealed that eEF1A1 is over expressed in gliomas (FIG. 4A) while eEF1A2 is down-regulated (FIG. 4B).

### Example 2

### New Pro-Drugs of Amaryllidaceae Isocarbostyril Derivatives Accumulating in the Brain and their Synthesis Routes

According to literature, the isocarbostyril natural products can be isolated from the corresponding Amaryllidaceae species which contain these natural products (Kornienko A et al., Chem Rev 2008; 108:1982-2014). The compounds of the invention and the subgroups thereof can be prepared from starting materials which are either commercially available or prepared by standard means obvious to those skilled in the art of organic chemistry or isolated from the Amaryllidaceae natural source.

More specifically, in order to synthesize the new prodrugs of general formula I (also called type I prodrugs or derivatives), two ways of synthesis are possible: In a first way, the 3,4-cyclic phosphate (3) derivative of the natural product is synthesized and then alkylated or substituted; or an activation/substitution of the phosphonate is performed to obtain the final product. In a second way, the dihydrogen phosphonate (7) is synthesised corresponding to the final product and then the final and desired product (4) is obtained starting from the natural product through cyclization. These two ways of synthesis are represented schematically below:

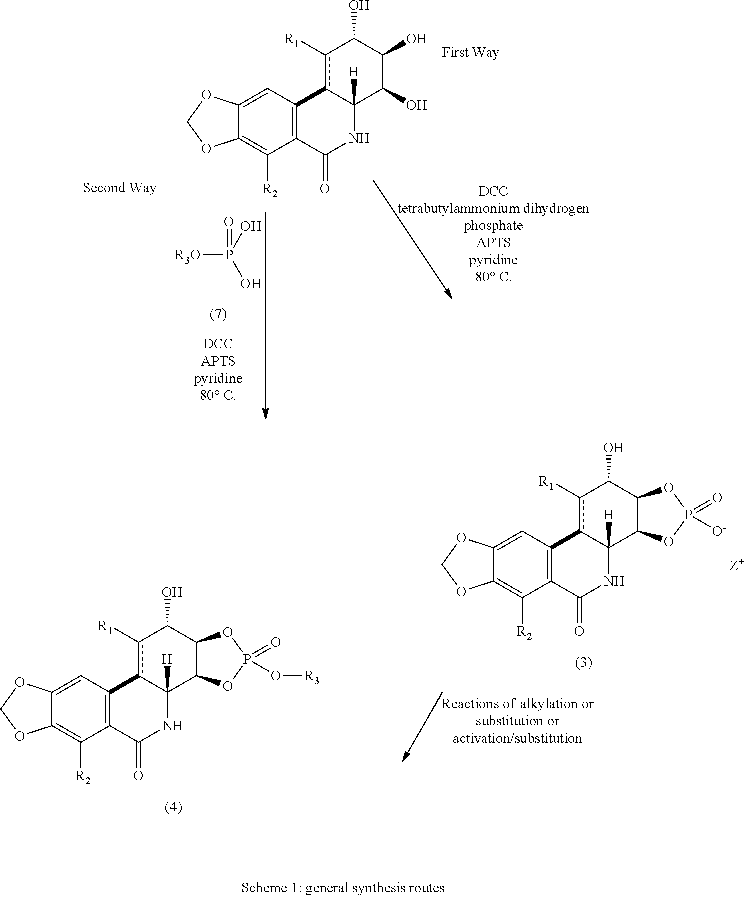

In order to prepare new Amaryllidaceae isocarbostyril prodrugs as defined herein, we used the known phosphate linker in position 3,4 of natural products as for example Narciclasine (cf. (3) defined by GR Pettit et al., also denominated narcistatin) as a starting point:

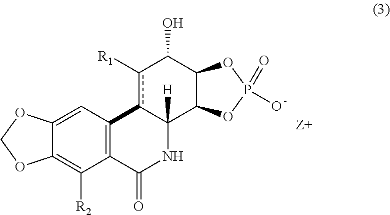

However, instead of rendering the molecule more hydrophilic, we link lipophilic chemical structures to it that will facilitate and increase the brain accumulation of the compound that will further free and deliver the natural isocarbostyril products as for example Narciclasine compound into the tumor bearing organ, i.e. the brain.

Specific examples of the Narciclasine and trans-dihydronarciclasine pro-drugs of the invention as defined by any one of the formulas described herein and their synthesis routes are described in detail herein:

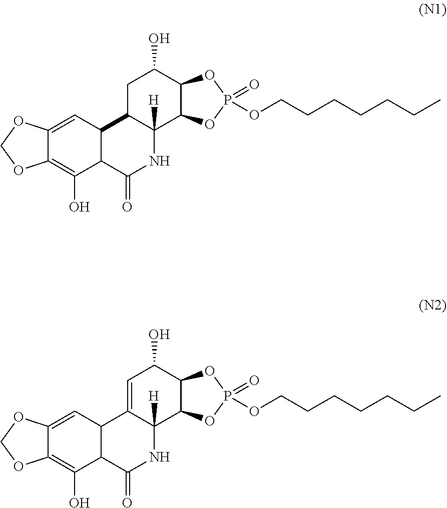

Compounds (N1) and (N2), which are based on general formulas (Ic) and (Id) having a linear C₇H₁₅alkyl group as the substituent for R₃ have a calculated log P value of 2.17 (+/−0.40) (Log P value calculated as in via http://www.vcclab.org/lab/alogps/start.html).

The synthesis route of the narcistatin derivative N2 is described below in Scheme 2. Starting from narciclasine, the 3,4-cyclic phosphate derivative of narciclasine is first synthesized by a step already performed by Pettit et al. (cf. Pettit et al., J. Nat. Prod. 2005, 68, 207-211). In a second step, the synthesized phosphate is alkylated in order to obtain the final derivative N2 (Scheme 2). The same synthesis route is used to obtain the N1 compound (single bond version).

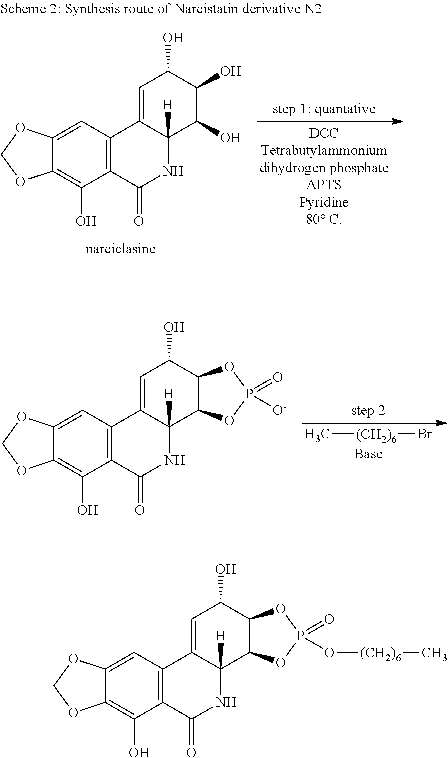

The experimental conditions are similar to those known for another phosphate and with benzyl bromide as alkylating agent (Burgada et al., II Phosphorus, Sulfur, Silicon and Related Elements 1987, 29, 275-282).

Alternatively, the compound of formula N2 can be obtained by the synthesis route depicted in Scheme 3 below. First, the monoheptylester phosphate is synthesized, starting from phosphoric acid and 1-heptanol, following a method already described with ethanol (Sorensen-Stowell et al., J. Org. Chem. 2005, 70, 4805-4809; Dueymes et al., Tetrahedron Lett. 2008, 49, 5300-530125, 26). In a second step, the monoheptylester phosphate reacts with Narciclasine and the compound of formula N2 is obtained.

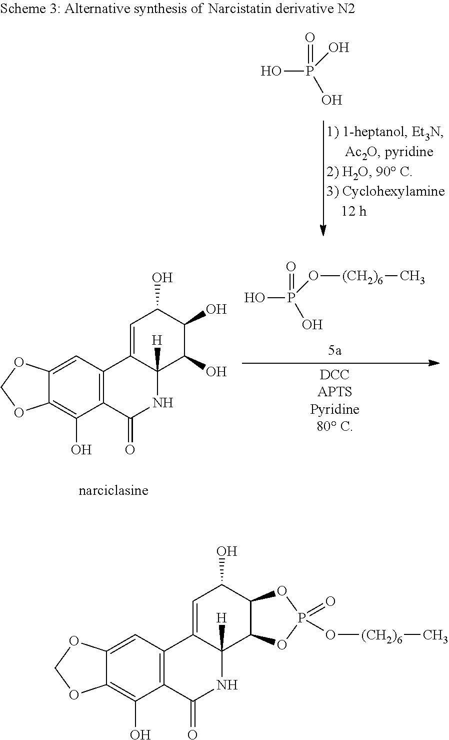

All compounds exemplified herein can generally be synthesised using the same reaction schemes, using the specific R3 groups. Further specific examples of Narciclasine and trans-dihydronarciclasine pro-drugs of the invention are:

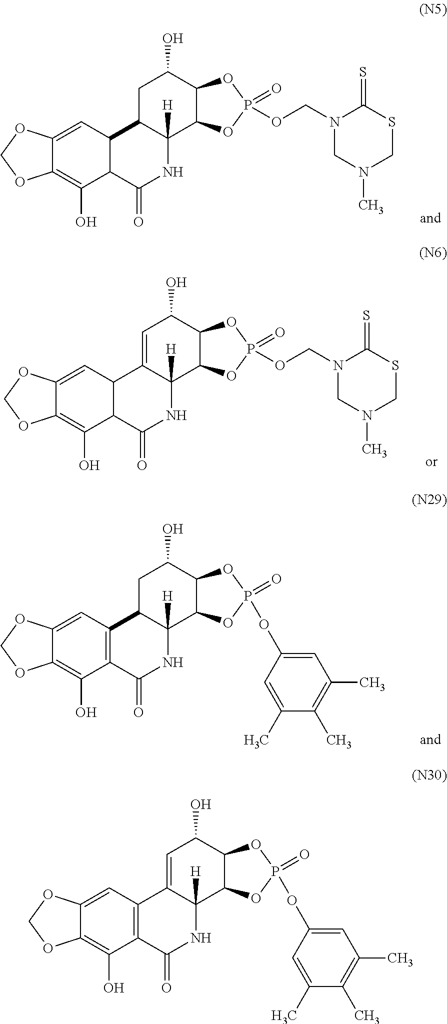

Compound N30 is e.g. synthesized according to Scheme 4. In this product, the heptyl chain of the N2 compound is replaced by a tri-methyl-phenyl moiety, resulting in a log P value of 2.19 (+/−0.43). The synthesis route is similar to the one of compound N2. Compound N29 (single bond version) is obtained following the same reaction scheme.

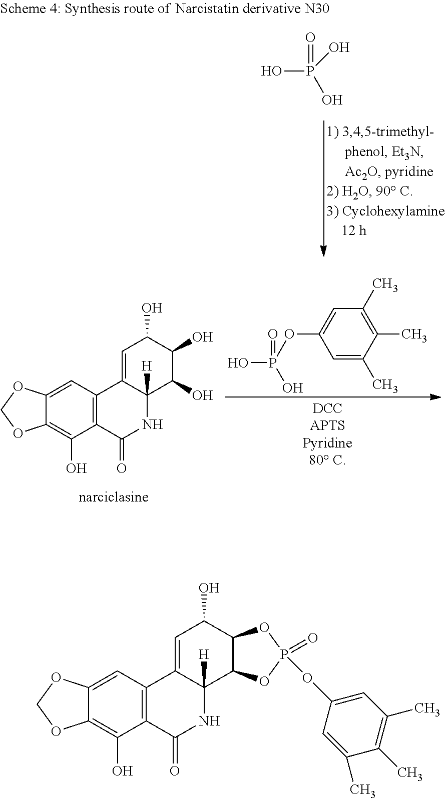

Alternatively, the N30 compound can be synthesized starting from the Narcistatin and commercial 3,4,5-trimethylphenol, using classical esterification of phosphates (Scheme 5).

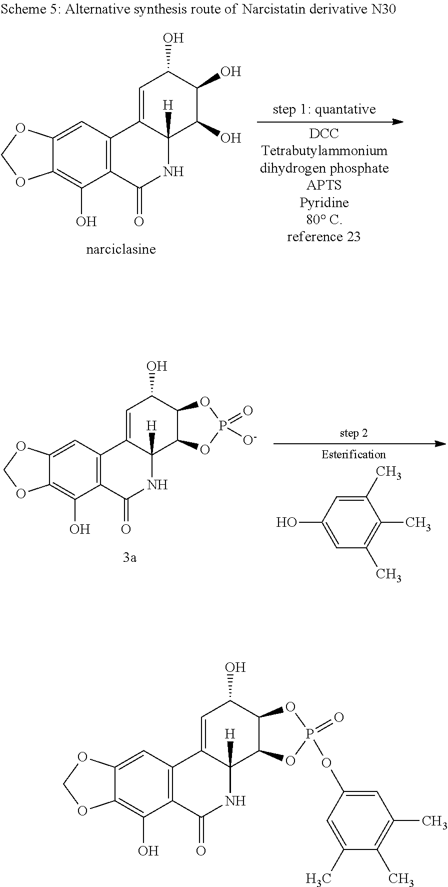

The same routes can be used to obtain the single bond version of N30, named N29.

Further specific examples of compounds that can be synthesized in a similar manner following schemes 2 and 3 are:

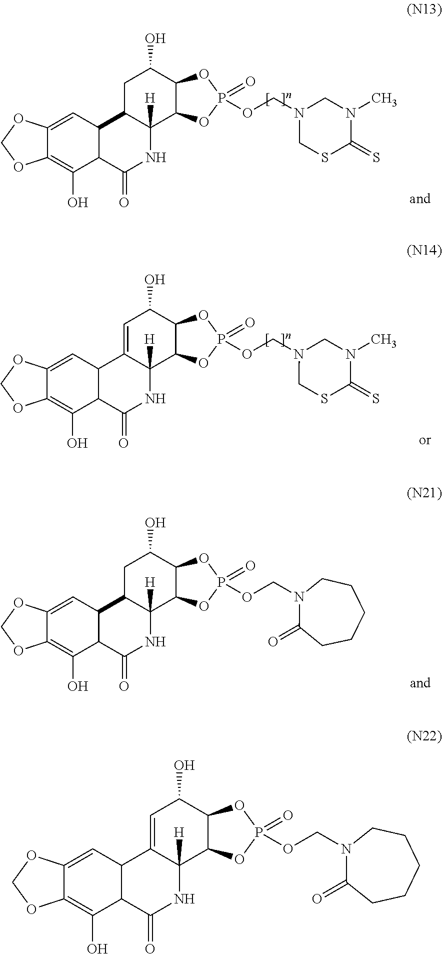

In order to synthesize the new prodrugs of general Formula II (also called type II prodrugs), we used one way of synthesis: the starting natural products were peracetylated and then a selective deprotection of the phenol function was performed. The corresponding phosphites were then coupled on the phenol of the Amaryllidaceae isocarbostyril derivatives in order to obtain the protected phosphate products. A deprotection of the acetate functions was then achieved to obtain the desired final products. This synthesis is represented schematically below:

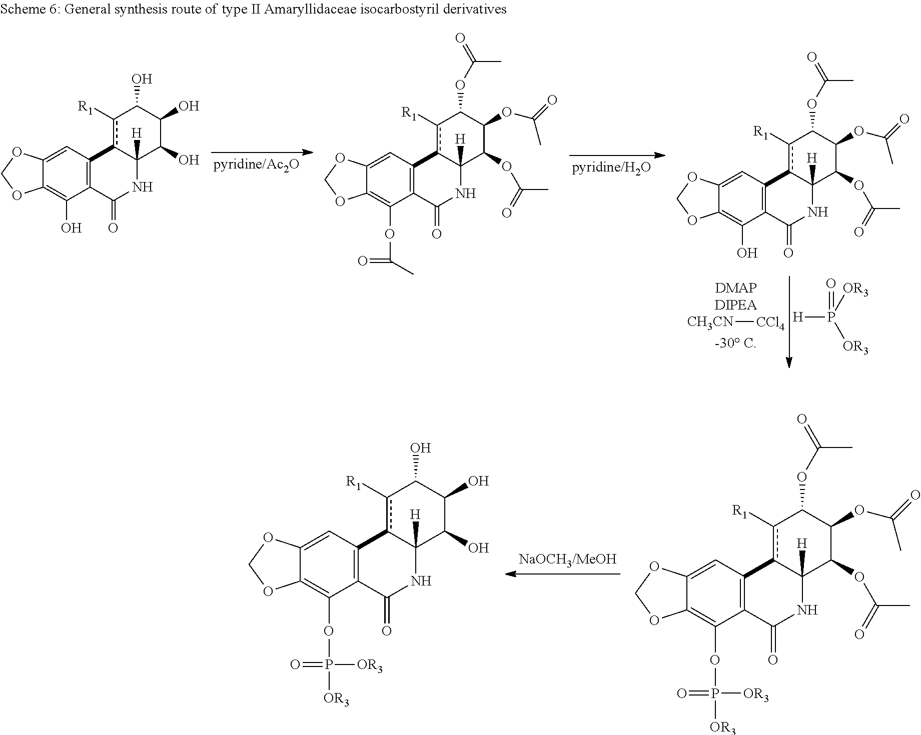

Compounds (N31) and (N32), which are based on general formulas (IIe) and (IIf) having a linear OC₄H₉ alkyl group as the substituent for R₃ have a calculated log P value of 1.51 (+/−0.39) and 1.44 (+/−0.47) (Log P value calculated as in via http://www.vcclab.org/lab/alogps/start.html).

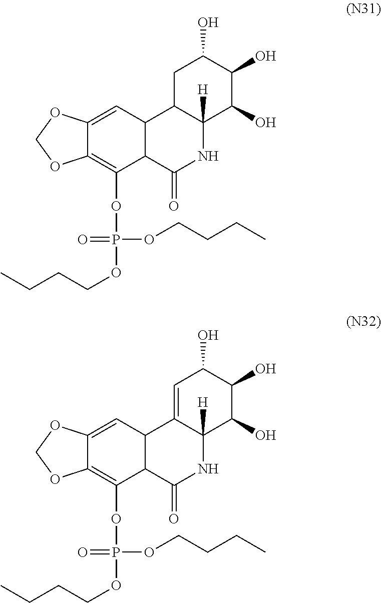

Compounds (N33) and (N34), which are based on general formulas (IIg) and (IIh) having a O-phenyl group as the substituent for R₃ have a calculated log P value of 2.13 (+/−0.44) and 2.08 (+/−0.38) (Log P value calculated as in via http://www.vcclab.org/lab/alogps/start.html).

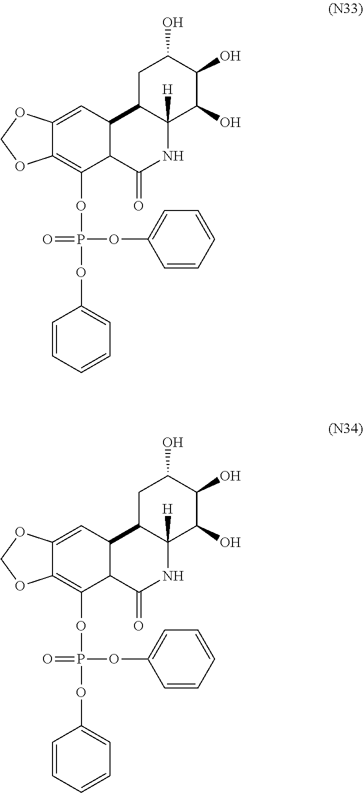

### Example 2

### Amaryllidaceae Isocarbostyril Derivatives as Novel Chemotherapeutic Weapons Against Brain Tumors

In vivo anti-cancer effects of Amaryllidaceae Isocarbostyril derivatives have been evaluated by the National Cancer Institute (NCI) in numerous studies (11 studies with narciclasine and 10 studies with pancratistatin) conducted with various mouse and human cancer models (NCI database). When looking at the results, it appears that significant anti-cancer effects (decrease in tumor size of >25% or increase in survival period>25%) could only be reached with chronic doses that are in fact toxic because they are higher than 1 mg/kg for rodents in the case of narciclasine (Ingrassia L et al, J Med Chem 2009; 52:1100-14). In all the studies reported by the NCI (i.e. available in the NCI database), tumors have been grafted subcutaneously, intra-peritoneally or intra-renally but none of those studies has been conducted in an intracranial tumor model. Moreover, the treatment administration route was mostly intra-peritoneal which is not clinically transposable regarding that this route of administration in human beings has been approved by the FDA in the oncology field only in the case of ovarian cancers for cisplatin administration.

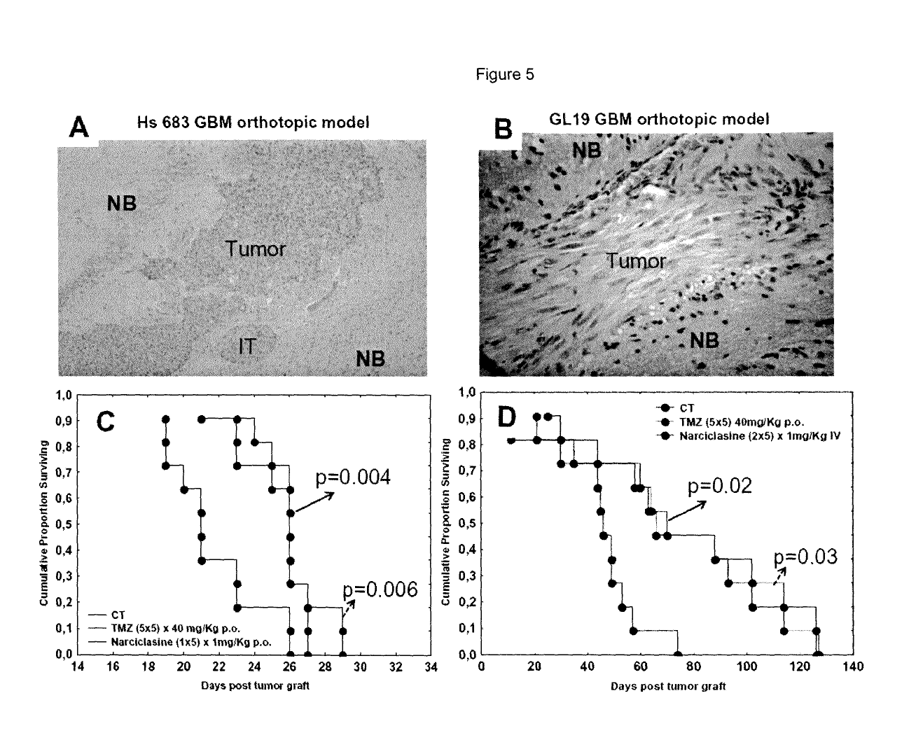

We evaluated the anti-cancer effects of Narciclasine at non toxic dose in two human glioblastoma (primary brain tumors) models (Hs683-model) that we grafted orthotopically directly into the brain of nude mice. Interestingly and unexpectedly, narciclasine given at 1 mg/kg once or twice per week during 5 consecutive weeks brought significant and comparable therapeutic benefits in terms of survival to temozolomide, the current FDA approved chemotherapeutical agent for those tumors that brings significant increase in survival of human glioma patients (Stupp R et al, N Engl J Med 2005 and Lancet Oncol 2009) (FIG. 5; Lefranc F et al, Mol Cancer Ther 2009). Please note that 1 mg/kg dose in rodents can be delivered 5 times per week during three weeks at least without any toxicity (Ingrasia L, J Med. Chem. 2009; 52:1100-14). The therapeutic schedule used here is even lower (one or two injections per week instead of 5 per week) and the administration route was intravenous or oral. The oral bioavailability of narciclasine has been evaluated at 32% meaning that the real dose administered in the case of Hs683 model is as weak as 0.32 mg/kg (Ingrassia I et al, J Med. Chem. 2009; 52:1100-14). The few promising NCI results have been obtained at doses higher than 1 mg/kg administered intraperitoneally or subcutaneously.

As the mechanism of action of Amaryllidaceae Isocarbostyrils involves a new target, i.e. eEF1A and is therefore completely different to the one of temozolomide (which is an alkylating agent leading to autophagy and apoptosis (Kanzawa T et al, Cell Death Differ 2004; Roos W P et al, Oncogene 2007)), we propose to use the claimed pro-drugs when glioblastoma patients treated with surgery followed by radiotherapy and temozolomide (i.e. the standard treatment) relapse.

Indeed, our aim is not to potentiate temozolomide anti-cancer effects because this later is sufficient till sensitivity is observed but to offer to brain tumor patients a new and completely different therapeutic weapon (see new target section) when the current available agents cannot help them anymore.

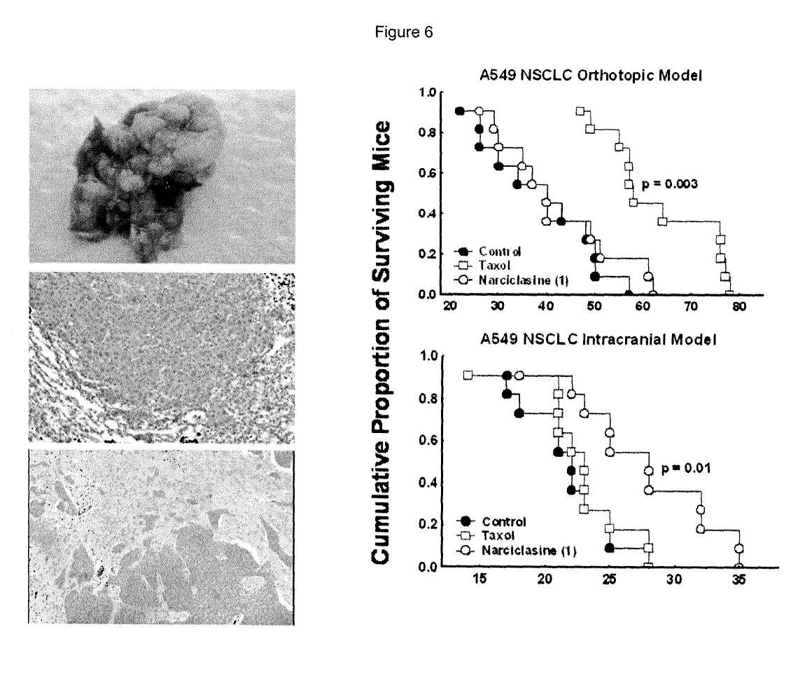

We tested also the anti-cancer effects of Narciclasine in non small cell lung carcinoma models (NSCLC). When human NSCLC A549 cells are grafted into the lungs of nude mice, those later develop lung tumors leading after to liver and brain metastases in 80% of the cases (Mathieu A et al, Cancer 2004). In this model, Narciclasine did not bring any increase in the survival of the animals while taxol, the reference drug for this cancer type, did significantly (FIG. 6; Ingrassia I et al, J Med. Chem. 2009; 52:1100-14). By contrast and against all odds, when those A549 cells are grafted directly into the brain of nude mice, Narciclasine was highly and significantly effective in terms of survival benefits while taxol became inefficient (FIG. 6; Ingrassia I et al, J Med. Chem. 2009; 52:1100-14).

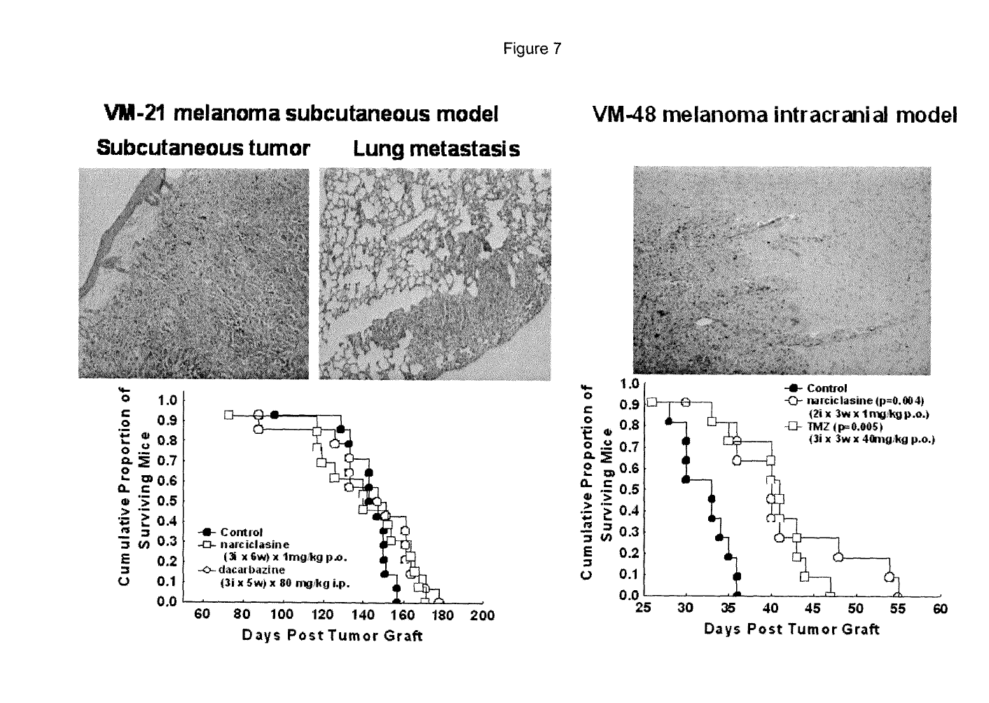

Similarly, we grafted subcutaneously human melanoma VM-21 cells. This graft leads to tumor formation and further lung metastases as illustrated in FIG. 7. In this model again, Narciclasine could not bring any survival benefit similar to that of dacarbazine, the only FDA approved cytotoxic chemotherapeutical agent for melanoma that however is known to have the very low response rate of 16% (Atallah E and Flaherty L, Curr Treat Options Oncol 2005). VM-48 cells have been isolated from a human brain metastasis of melanoma. We grafted those cells in their original environment, i.e. the brain to mimic human melanoma brain metastasis (FIG. 7).

Again, against all odds, Narciclasine given intravenously at the non toxic dose of 1 mg/kg twice a week during 3 consecutive weeks brought a significant increase in terms of animal survival that was similar to the one brought by temozolomide (literature data indicate that temozolomide is at least as effective than dacarbazine for metastatic melanoma treatment (Middelton M R et al, J Clin Oncol 2000) but could be preferred in the case of melanoma brain metastases (Raizer J J et al, Neuro Oncol 2008)).

Those results and the previous ones in glioblastoma models led us to postulate i) that Narciclasine and other Amaryllidaceae isocarbostyril lipohilic pro-drugs should be developed as novel anti-cancer drugs to combat brain tumors at non toxic doses and ii) that Narciclasine-induced therapeutic benefits in brain tumors could relate, at least partly to the Narciclasine target we recently evidenced, i.e. eEF1A which expression is deregulated in those tumors. For this issue, we aimed to enhance Narciclasine activity against brain tumors thanks to the development of lipophilic pro-drugs of this compound, or of any Amaryllidaceae isocarbostyril derivatives, that will enhance the blood brain barrier passage as well as its accumulation into the brain.

### Example 3

### Quantification of Narciclasine Concentration in the Brain

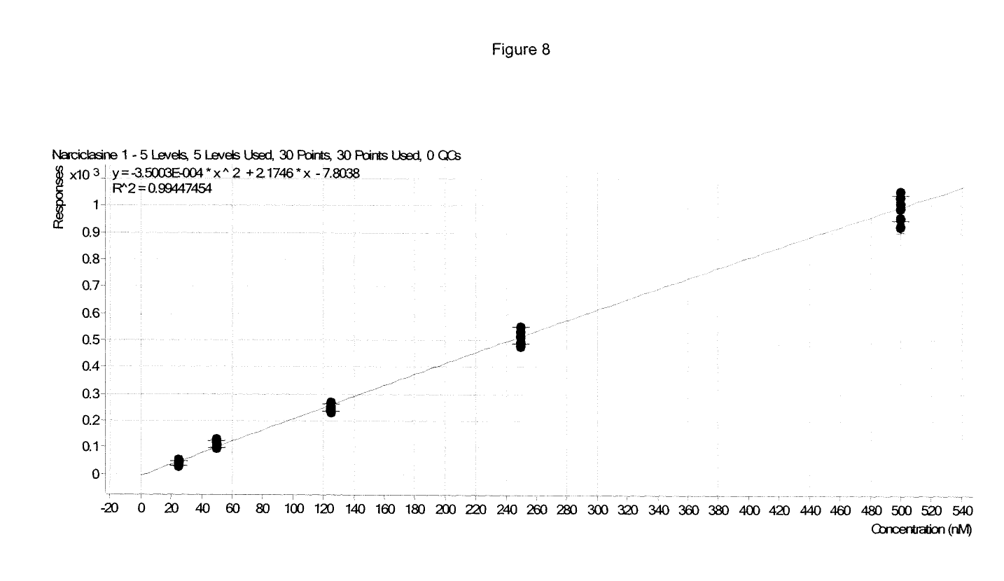

Simple quantification of Narciclasine concentration can be made through fluorescent method. However for quantification in complex fluids and organ tissues, we developed an analytical method to be reproducible and quantitative. We made use of quantitative mass spectra analysis in this aim (LC/MS-MS analysis, Q-TOFF). We first made a standard curve for Narciclasine (FIG. 8) and an internal standard (in this case cytochalasin B; data not shown).

Narciclasine calibration curve

| | cc | | | |
|---|---|---|---|---|
| | nM | Mean response | SD | vc % |
| Std_1 | 25 | 41 | 9 | 21.51 |
| Std_2 | 50 | 112 | 13 | 11.78 |
| Std_3 | 125 | 248 | 13 | 5.13 |
| Std_4 | 250 | 518 | 30 | 5.87 |
| Std_5 | 500 | 991 | 38 | 3.88 |

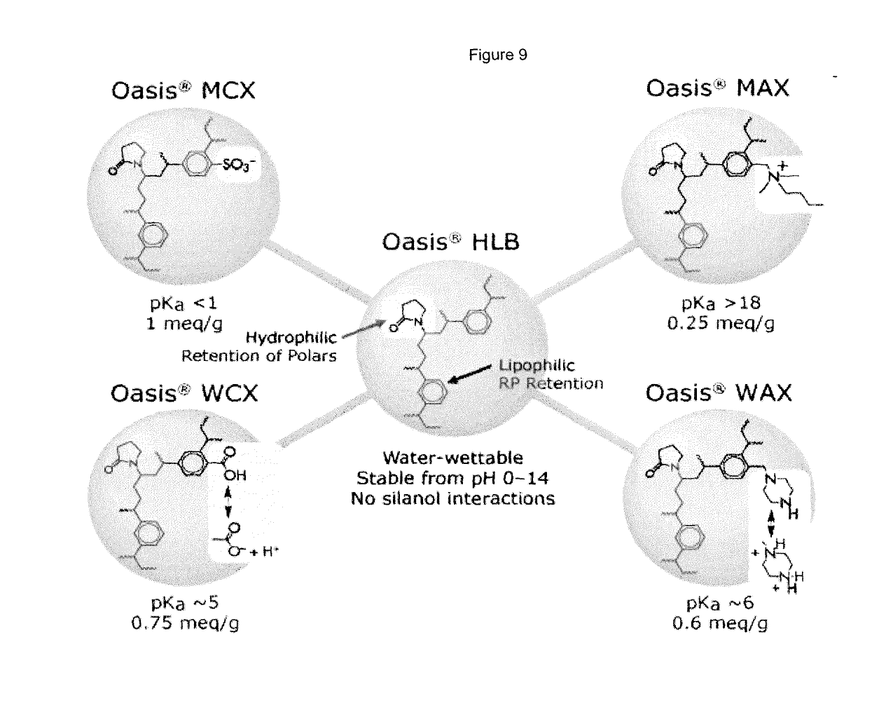

We used Solid Phase Extraction columns (SPE) to purify biological samples, in particular plasmatic ones. This column contains HLB silica (FIG. 9) which is characterized by hydrophilic properties thanks to N-vinylpyrrolidone groups and lipophilic properties thanks to divinylbenzene groups (FIG. 9, center). This column is thus able to fix Narciclasine and the internal standard chosen (here cytochalasin B by e.g.) while proteins will be eluted by successive wash. Before the loading of the sample, the column has to be activated (1 ml MeOH) and stabilized (1 ml water). The sample containing the compounds to be fixed will then be injected.

TABLE 2
results obtained by LC-MS in plasmatic samples. Bold values are the best results
obtained with this method. The method is not sensitive enough of concentrations < to 20 nM.

| | | 50 nM | 100 nM | 200 nM | 400 nM |
|---|---|---|---|---|---|
| [symbol] mple solutuion | Narciclasine | 66 | 179 | 290 | 501 |

| | | Time (min) | | | | | | | | | | | |
|---|---|---|---|---|---|---|---|---|---|---|---|---|---|
| | | T0 | T30 | T60 | T0 | T30 | T60 | T0 | T30 | T60 | T0 | T30 | T60 |
| Plasma | Narciclasine | 35 | 50 | 48 | 62 | 88 | 95 | 152 | 141 | 195 | 248 | 366 | 250 |

[symbol]

indicates data missing or illegible when filed

The sample contains plasma, the compound of interest (e.g. Narciclasine) and an internal standard (e.g. cytochalasin B) in a 50:50 mixture of water and MeOH. We add to this preparation 1% of formic acid to break links between our molecule of interest and plasmatic proteins.

We evaluated 3 incubation times to allow formic acid to act: t=0, t=30 min, t=60 min. The sample is loaded onto the column to allow penetration and fixation of the compounds. Proteins are eluted by wash with 5% MeOH solution in water. Recovery of the compound is obtained by elution with 100% MeOH solution.

Results are illustrated in Table 2.

This method can be adapted to quantify the content of the Amaryllidaceae isocarbostyril derivative in various organ tissues and in the brain in particular in rodents (mice and/or rats). Tissues are minced, syringed and digested by enzymes to form solutions that will be further handled in a similar way than the plasmatic samples to evaluate organ accumulation and retention time of the different prodrugs of the invention. To first validate the method in tissues, fresh tissue solutions of various organs are first prepared that will be “contaminated” exogenously by various Narciclasine concentrations to assess accuracy of the method.

Mice will receive 1 to 5 consecutive intravenous injections of 1 mg/kg Narciclasine or one of the prodrugs of the invention or left untreated. Blood, brain, lung, liver and kidney will be collected after time periods ranging from 5 min to 30 h after the last injection. 200 mg per organ will be minced and prepared for Narciclasine measurement and compared to plasmatic concentration.

The same experiment can then be carried out in mice grafted with tumor cells as described above, to evaluate efficacy of anti-tumor effects of the different prodrugs of the invention in comparison to Narciclasine.

1. A pro-drug of Amaryllidaceae isocarbostyril derivatives that is lipophilic, defined by having a log P value≧1.5 and <3, preferably of about 2.

2. The pro-drug of Amaryllidaceae isocarbostyril derivatives of claim 1, defined by the following general formula (I): 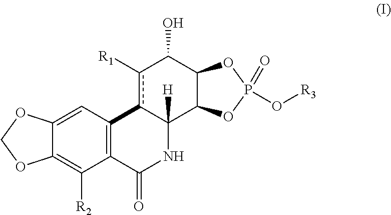
wherein the dotted line can be a single or double bond,
R₁ is selected from the group consisting of: H, OH, OR₄, NR₅R₆, alkyl, aryl, cycloC₁-C₆alkyl, and cyclohexyl;
R₂ is selected from the group consisting of: H, and OR₇;
R₃ is selected from the group consisting of: a substituted or non-substituted straight or branched chain C₁-C₂₄alkyl group, a substituted or non-substituted aryl group, preferably substituted or non-substituted C₆-C₁₈aryl group, a substituted or non-substituted heteroaryl group, a substituted or non-substituted arylalkyl group, preferably a C₆-C₁₈arylC₁-C₆alkyl group, a cycloC₁-C₆alkyl group, a polyoxyalkylene chain of the formula (CxHyO)p, where x is 3 or more carbon atoms, p is 2 or more, and y is from 2x−2 to 2x, a heterocyclyl-C₁-C₂₄alkyl group or a heteroaryl-C₁-C₂₄alkyl, a —C₃-C₂₄alkynyl group, and a —C₃-C₂₄alkenyl group;
R₄, R₅, R₆ and R₇ are each independently selected from the group consisting of: H, C₁-C₆alkyl, C₆-C₁₈aryl, carbonyl-C₁-C₆alkyl; and carbonyl-C₆-C₁₀aryl;
each group optionally being substituted by a C₁-C₆-alkyl, OXO or ═S.

3. The pro-drug Amaryllidaceae isocarbostyril derivatives according to claim 2, defined by the following general formulas (Ia) or (Ib): 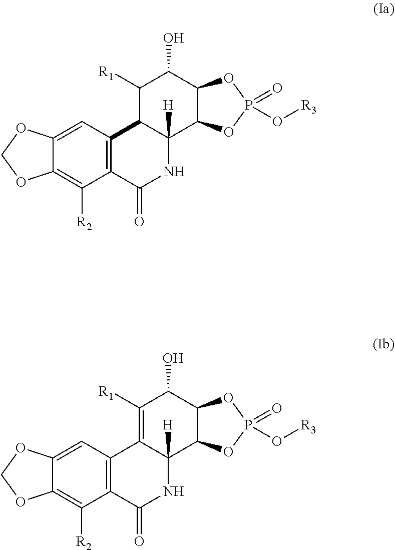
wherein in any one of the formulas (Ia) or (Ib):
R₁ is selected from the group consisting of: H, OH, OR₄, NR₅R₆, alkyl, aryl, cycloC₁-C₆alkyl, and cyclohexyl;
R₂ is selected from the group consisting of: H, and OR₇;
R₃ is selected from the group consisting of: a substituted or non-substituted straight or branched chain C₁-C₂₄alkyl group, a substituted or non-substituted aryl group, preferably substituted or non-substituted C₆-C₁₈aryl group, a substituted or non-substituted heteroaryl group, a substituted or non-substituted arylalkyl group, preferably a C₆-C₁₈arylC₁-C₆alkyl group, a cycloC₁-C₆alkyl group, a polyoxyalkylene chain of the formula (CxHyO)p, where x is 3 or more carbon atoms, p is 2 or more, and y is from 2x−2 to 2x, a heterocyclyl-C₁-C₂₄alkyl group or a heteroaryl-C₁-C₂₄alkyl, a —C₃-C₂₄alkynyl group, and a —C₃-C₂₄alkenyl group;
R₄, R₅, R₆ and R₇ are each independently selected from the group consisting of: H, C₁-C₆alkyl, C₆-C₁₈aryl, carbonyl-C₁-C₆alkyl; and carbonyl-C₆-C₁₀aryl;
each group optionally being substituted by a C₁-C₆-alkyl, OXO or ═S.

4. The pro-drug according to claim 2, defined by the following general formula (Ic) or (Id): 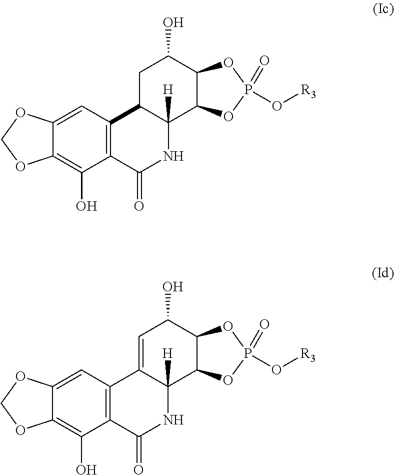
wherein
R₃ is selected from the group consisting of: straight or branched chain C₁-C₂₄alkyl, C₆-C₁₈aryl, C₆-C₁₈arylC₁-C₆alkyl, cycloC₁-C₆alkyl, a polyoxy-C₃-C₂₄alkylene chain of the formula (CxHyO)p, where x is 3 or more carbon atoms, p is 2 or more, and y is from 2x−2 to 2x, a heterocyclyl-C₁-C₂₄alkyl or heteroaryl-C₁-C₂₄alkyl, C₃-C₂₄alkynyl, and C₃-C₂₄alkenyl;
R₄, R₅, R₆ and R₇ are each independently selected from the group consisting of: H, C₆-C₁₈aryl, carbonyl-C₁-C₆alkyl; and carbonyl-C₆-C₁₀aryl;
each group optionally being substituted by a C₁-C₆-alkyl, *=O (OXO-group) or *=S (thione group), wherein the asterisk (*) is used herein to indicate the point at which a mono- or bivalent radical depicted is connected to the structure to which it relates and of which the radical forms part.

5. The pro-drug according to claim 2, wherein R₃=a linear C₁-C₂₄-alkyl, such as n-Methyl, n-Ethyl, n-Propyl, n-Butyl, n-Pentyl, n-Hexyl, n-Heptyl, n-Octyl, n-Nonyl, n-Decyl, n-Undecyl, n-Dodecyl.

6. The pro-drug according to claim 2, wherein R₃ is C₇H₁₅ as in Formula (Ie) and (If): 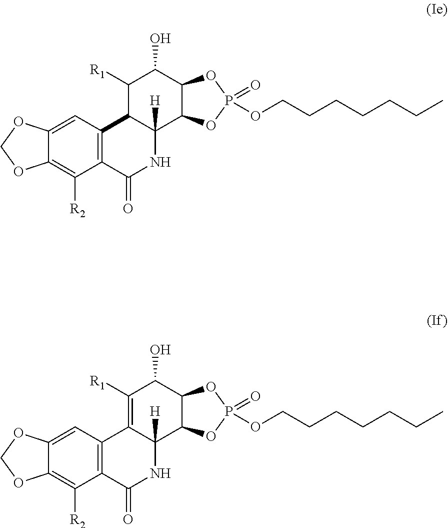
wherein
R₁ and R₂ are each independently selected from the group consisting of: H, OH, and O—C₁-C₆alkyl.

7. The pro-drug according to claim 2, defined by the following general formula (Ig) or (Ih): 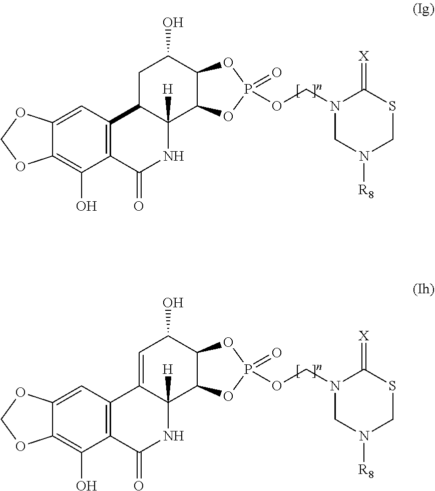
wherein
X is selected from O or S;
n is an integer selected from: 1, 2, 3, 4, 5, 6, 7, 8, 9, 10, 11, 12, 13, 14, 15, 16, 17, 18, 19, 20, 21, 22, 23, or 24;
R₈ is selected from the group consisting of: H and C₁-C₆alkyl.

8. The pro-drug according to claim 2, defined by the following general formula (Ii) or (Ij): 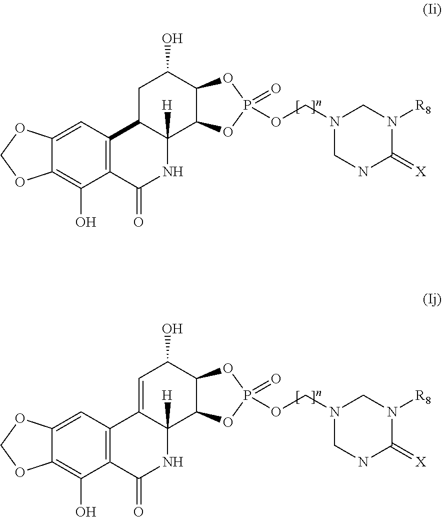
wherein
X is selected from O or S;
n is an integer selected from: 1, 2, 3, 4, 5, 6, 7, 8, 9, 10, 11, 12, 13, 14, 15, 16, 17, 18, 19, 20, 21, 22, 23, or 24;
R₈ is selected from the group consisting of: H and C₁-C₆alkyl.

9. The pro-drug according to claim 2, defined by the following general formula (Ik) or (Il): 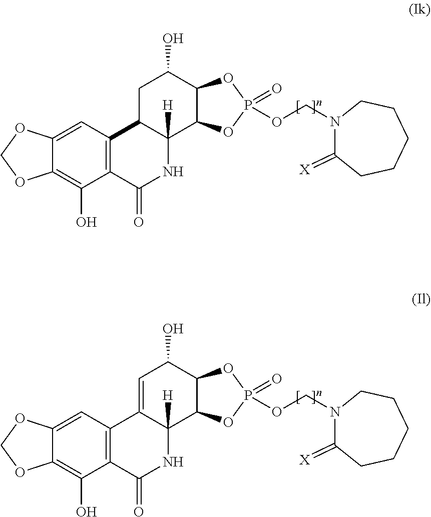
wherein
X is selected from O or S;
n is an integer selected from: 1, 2, 3, 4, 5, 6, 7, 8, 9, 10, 11, 12, 13, 14, 15, 16, 17, 18, 19, 20, 21, 22, 23, or 24.

10. The pro-drug of Amaryllidaceae isocarbostyril derivatives of claim 1, defined by the following general formula (II): 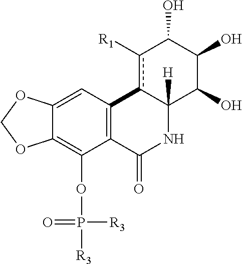
wherein the dotted line can be a single or double bond;
R₁ is selected from the group consisting of: H, OH, OR₄, NR₅R₆, alkyl, aryl, cycloC₁-C₆alkyl, and cyclohexyl;
R₃ is independently selected from the group consisting of: H, OH, a substituted or non-substituted straight or branched chain O—C₁-C₂₄alkyl, a substituted or non-substituted O-aryl group, preferably substituted or non-substituted O—C₆-C₁₈aryl, a substituted or non-substituted O-heteroaryl group, a substituted or non-substituted O-aryl-alkyl group, preferably a O—C₆-C₁₈arylC₁-C₆alkyl group, a oxy-cycloC₁-C₆alkyl group, a O-polyoxyalkylene chain of the formula O—(CxHyO)p, where x is 3 or more carbon atoms, p is 2 or more, and y is from 2x−2 to 2x, a O-heterocyclyl-C₁-C₂₄alkyl group, a O-heteroaryl-C₁-C₂₄alkyl group, a O—C₃-C₂₄alkynyl group, and a O—C₃-C₂₄alkenyl group;
wherein R₄, R₅, R₆ and R₇ are each independently selected from the group consisting of: H, C₁-C₆alkyl, C₆-C₁₈aryl, carbonyl-C₁-C₆alkyl; and carbonyl-C₆-C₁₀aryl;
each group optionally being substituted by a C₁-C₆-alkyl, OXO or ═S.

11. The pro-drug of Amaryllidaceae isocarbostyril derivatives of claim 10, defined by the following general formulas (IIa) or (IIb): 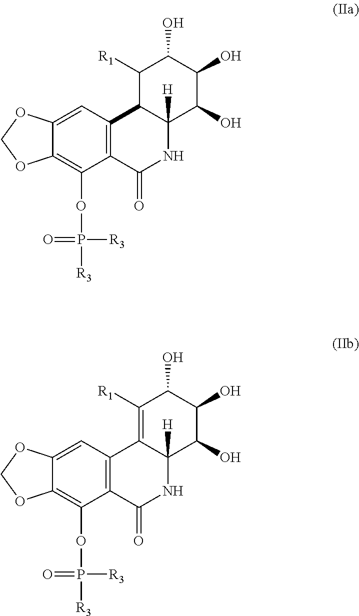
wherein in any one of the formulas II, IIa or IIb:
R₁ is selected from the group consisting of: H, OH, OR₄, NR₅R₆, alkyl, aryl, cycloC₁-C₆alkyl, and cyclohexyl;
R₃ is independently selected from the group consisting of: H, OH, a substituted or non-substituted straight or branched chain O—C₁-C₂₄alkyl, a substituted or non-substituted O-aryl group, preferably substituted or non-substituted O—C₆-C₁₈aryl, a substituted or non-substituted O-heteroaryl group, a substituted or non-substituted O-aryl-alkyl group, preferably a O—C₆-C₁₈arylC₁-C₆alkyl group, a oxy-cycloC₁-C₆alkyl group, a O-polyoxyalkylene chain of the formula O—(CxHyO)p, where x is 3 or more carbon atoms, p is 2 or more, and y is from 2x−2 to 2x, a O-heterocyclyl-C₁-C₂₄alkyl group, a O-heteroaryl-C₁-C₂₄alkyl group, a O—C₃-C₂₄alkynyl group, and a O—C₃-C₂₄alkenyl group;
wherein R₄, R₅, R₆ and R₇ are each independently selected from the group consisting of: H, C₁-C₆alkyl, C₆-C₁₈aryl, carbonyl-C₁-C₆alkyl; and carbonyl-C₆-C₁₀aryl;
each group optionally being substituted by a C₁-C₆-alkyl, OXO or ═S.

12. The pro-drug of Amaryllidaceae isocarbostyril derivatives of claim 10, defined by the following general formula (IIc) or (IId): 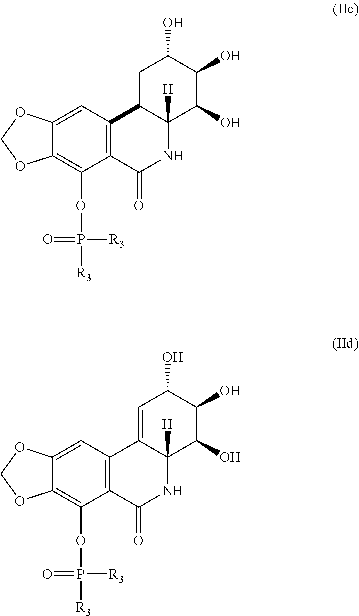
wherein
R₃ is independently selected from the group consisting of: H, OH, a substituted or non-substituted straight or branched chain O—C₁-C₂₄alkyl, a substituted or non-substituted O-aryl group, preferably substituted or non-substituted O—C₆-C₁₈aryl, a substituted or non-substituted O-heteroaryl group, a substituted or non-substituted O-aryl-alkyl group, preferably a O—C₆-C₁₈arylC₁-C₆alkyl group, a oxy-cycloC₁-C₆alkyl group, a O-polyoxyalkylene chain of the formula O—(CxHyO)p, where x is 3 or more carbon atoms, p is 2 or more, and y is from 2x−2 to 2x, a O-heterocyclyl-C₁-C₂₄alkyl group, a O-heteroaryl-C₁-C₂₄alkyl group, a O—C₃-C₂₄alkynyl group, and a O—C₃-C₂₄alkenyl group;
wherein R₄, R₅, R₆ and R₇ are each independently selected from the group consisting of: H, C₁-C₆alkyl, C₆-C₁₈aryl, carbonyl-C₁-C₆alkyl; and carbonyl-C₆-C₁₀aryl;
each group optionally being substituted by a C₁-C₆-alkyl, *=O (OXO-group) or *=S (thione group).

13. The pro-drug of Amaryllidaceae isocarbostyril derivatives of claim 10, wherein R₃ is selected from: H, OH, a linear O—C₁-C₂₄-alkyl, such as O-n-Methyl, O-n-Ethyl, O-n-Propyl, O-n-Butyl, O-n-Pentyl, O-n-Hexyl, O-n-Heptyl, O-n-Octyl, O-n-Nonyl, O-n-Decyl, O-n-Undecyl, or O-n-Dodecyl.

14. The pro-drug of Amaryllidaceae isocarbostyril derivatives of claim 13, wherein R₃ is O—C₄H₉ as in Formula (IIe) and (IIf): 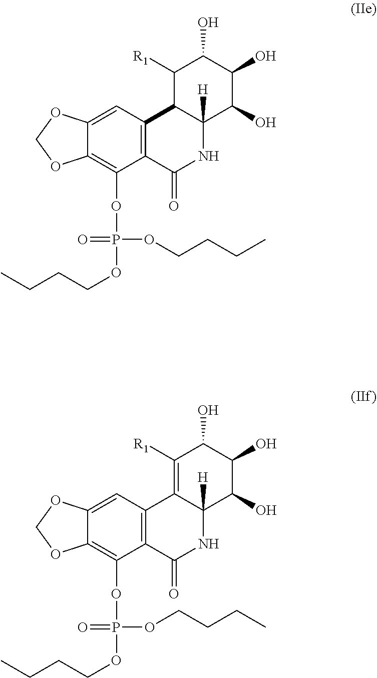
wherein R₁ is selected from the group consisting of: H, OH, and O—C₁-C₆alkyl.

15. The pro-drug of Amaryllidaceae isocarbostyril derivatives of claim 13, wherein R₃ is O-aryl and wherein R₁ is selected from the group consisting of: H, OH, and O—C₁-C₆alkyl.

16. The pro-drug of Amaryllidaceae isocarbostyril derivatives of claim 13, wherein R₃ is O-phenyl as in Formula (IIg) and (IIh): 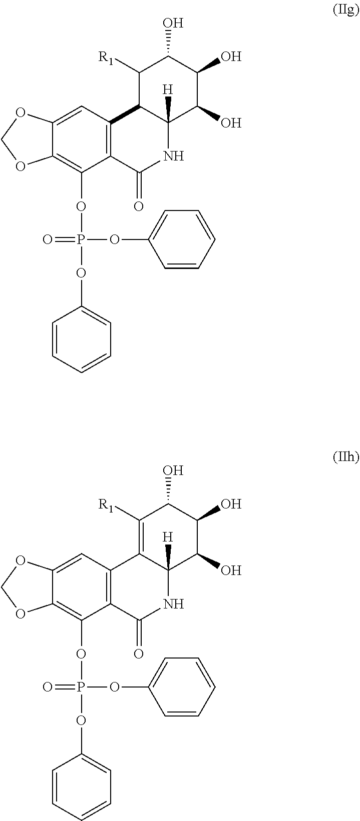
wherein R₁ is selected from the group consisting of: H, OH, and O—C₁-C₆alkyl.

17. The pro-drug of Amaryllidaceae isocarbostyril derivatives of claim 10, wherein R₁ is hydrogen.

18. The pharmaceutically acceptable addition salts, hydrates or solvates and/or stereoisomers, tautomers or diastereoisomers of any one of the Narciclasine pro-drugs according to claim 1.

19-20. (canceled)

21. A pharmaceutical composition comprising a pro-drug according to claim 1, or the pharmaceutically acceptable addition salts, hydrates or solvates thereof and/or their stereoisomers, tautomers or diastereoisomers.

22. A method for treating brain tumors or brain cancer which comprises administering a medicament or pharmaceutical composition comprising at least one pro-drug according to claim 1, or the pharmaceutically acceptable addition salts, hydrates or solvates thereof and/or their stereoisomers, tautomers or diastereoisomers as an active ingredient to an individual in need thereof, whereby the cancer is treated.

23. A kit for use in treating brain tumors or brain cancer and related disorders in an individual in need thereof comprising a therapeutically effective amount of the pharmaceutical composition comprising at least one pro-drug according to claim 1, or the pharmaceutically acceptable addition salts, hydrates or solvates thereof and/or their stereoisomers, tautomers or diastereoisomers as an active ingredient, optionally in combination with a pharmaceutically acceptable carrier.

24. A method of screening to identify new anti-cancer agents targeting eEF comprising the steps of:
a) contacting a candidate anti-cancer agent with the eEF receptor,
b) evaluating whether or not said candidate anti-cancer agent binds the eEF1A receptor, and
c) optionally analyzing the effect of the candidate agent on the activity of said eEF1A receptor.

25. The method according to claim 24, wherein the activity of the receptor is evaluated by analyzing the growth or progression of said tumor cell.
